(19) United States
(12) Patent Application Publication
(10) Pub. No.: US 2003/0069423 A1
(43) Pub. Date: Apr. 10, 2003

(54) Novel processes for the preparation of adenosine compounds and intermediates thereto

(76) Inventors: Timothy A. Ayers, Bridgewater, NJ (US); Geoffrey A. D'Netto, Hillsborough, NJ (US); Gregory G. Kubiak, Easton, PA (US); James J. Mencel, North Wales, PA (US); Michael K. O'Brien, Berwyn, PA (US); Matthew R. Powers, Barto, PA (US); John J. Shay, Bethlehem, PA (US); Adam W. Sledeski, Belle Mead, NJ (US); David S. Teager, Marcus Hook, PA (US); Benoit Vanasse, New Hope, PA (US)

Correspondence Address: ROSS J. OEHLER, AVENTIS PHARMACEUTICALS INC., ROUTE 202-206, MAIL CODE: D-303A, BRIDGEWATER, PA, 08807, US

(21) Appl. No.: 10/143,611

(22) Filed: May 10, 2002

Related U.S. Application Data
(60) Provisional application No. 60/289,926, filed on May 10, 2001.

(51) Int. Cl. C07D 473/34; C07D 471/02; A61K 31/52

(52) U.S. Cl. 544/277; 514/210.21; 514/263.2; 514/263.22; 514/263.24; 546/118; 514/303

(57) ABSTRACT

Novel processes for the preparation of adenosine compounds and intermediates thereto. The adenosine compounds prepared by the present processes may be useful as cardiovascular agents, more particularly as antihypertensive and anti-ischemic agents, as cardioprotective agents which ameliorate ischemic injury or myocardial infarct size consequent to myocardial ischemia, and as an antilipolytic agents which reduce plasma lipid levels, serum triglyceride levels, and plasma cholesterol levels. The present processes may offer improved yields, purity, ease of preparation and/or isolation of intermediates and final product, and more industrially useful reaction conditions and workability.

This application is entitled to the benefit of earlier filed U.S. Provisional Application No. 60/289,926, filed May 10, 2001.

### FIELD OF THE INVENTION

The present invention relates to novel processes for the preparation of adenosine compounds. More particularly, the present invention relates to novel processes for the preparation of adenosine compounds and analogs thereof, and intermediates to such compounds.

### BACKGROUND OF THE INVENTION

Adenosine compounds, as exemplified, for example, by (1R,2S,3R,5R)-5-methoxymethyl-3-[6-[1-(5-trifluoromethyl-pyridin-2-yl)pyrrolidin-3(S)-ylamino]-purin-9-yl]cyclopentane-1,2-diol], may be useful as cardiovascular agents, more particularly as antihypertensive and anti-ischemic agents, as cardioprotective agents which ameliorate ischemic injury or myocardial infarct size consequent to myocardial ischemia, and as an antilipolytic agents which reduce plasma lipid levels, serum triglyceride levels, and plasma cholesterol levels. See, e.g., WO 98/01426. Methods for the preparation of these adenosine compounds and intermediates thereto are also disclosed, for example, in WO 98/01426.

Prior art methods may be effective for the preparation of adenosine compounds, particularly on a smaller scale, for example, from about gram to about kilogram quantities. However, improved methods may be needed for the preparation of adenosine compounds on a larger scale, including commercial scale, for example, from about multi-kilogram up to about ton quantities or more of compound. This is because prior art methods for the preparation of adenosine compounds are generally multi-step processes, and typically involve isolation and/or purification of the various intermediates to the adenosine compounds. While these isolation and purification steps may be readily performed on a smaller scale, they may be problematic in large scale syntheses due to, for example, the additional processing steps involved and the added cost associated therewith. In addition, certain of the adenosine compounds and/or intermediates thereto may not be crystalline materials, such that isolation of the compounds and/or intermediates thereto may necessitate, for example, removal of reaction and/or work-up solvents in vacuo. While such isolation steps may be appropriate when working on a smaller scale, they may not be feasible on a larger scale, particularly when the solvents employed have elevated boiling points.

Accordingly, new and/or better alternatives to prior art methods for the preparation of adenosine compounds, particularly on a larger scale, are needed. The present invention is directed to this, as well as other important ends.

### SUMMARY OF THE INVENTION

Accordingly, the present invention is directed, in part, to novel processes for preparing adenosine compounds. Specifically, in one embodiment, there are provided processes for the preparation of a compound of formula (I):

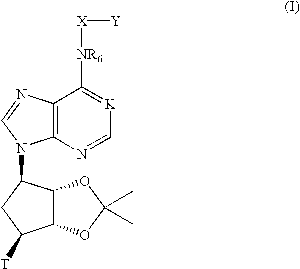

wherein:

K is N, N→O or CH;

R₆ is hydrogen, alkyl, allyl, 2-methylallyl, 2-butenyl or cycloalkyl;

X is

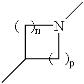

where the nitrogen of the ring of X is substituted by Y;

Y is hydrogen, alkyl, optionally substituted aralkyl, optionally substituted aryl, optionally substituted heterocyclyl or optionally substituted heterocyclylalkyl;

n and p are independently 0, 1, 2 or 3, provided that the sum of n and p is at least 1;

T is hydrogen, alkyl, acyl, thioacyl, halo, carboxyl, N(R₁)(R₂)C(═O), N(R₁)(R₂)C(═S) or R₃O—CH₂;

R₁, R₂ and R₃ are independently hydrogen, alkyl or cycloalkyl;

comprising:

contacting a compound of formula (II)

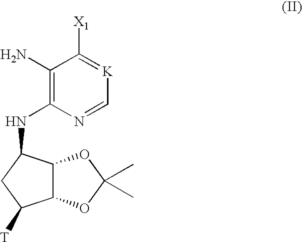

where X₁ is halo; with a formic acid derivative to provide a compound of formula (III):

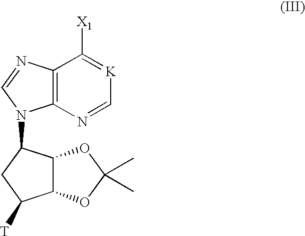

and contacting the compound of formula (III) with a compound of formula (IV)

R₆NH—X—Y (IV)

to provide the compound of formula (I) or a pharmaceutically acceptable salt form thereof.

Another embodiment relates to processes for the preparation of a compound of formula (V):

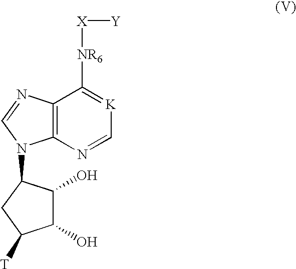

wherein:

K is N, N→O or CH;

R₆ is hydrogen, alkyl, allyl, 2-methylallyl, 2-butenyl or cycloalkyl;

X is

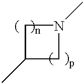

where the nitrogen of the ring of X is substituted by Y;

Y is hydrogen, alkyl, optionally substituted aralkyl, optionally substituted aryl, optionally substituted heterocyclyl or optionally substituted heterocyclylalkyl;

n and p are independently 0, 1, 2 or 3, provided that the sum of n and p is at least 1;

T is hydrogen, alkyl, acyl, thioacyl, halo, carboxyl, N(R₁)(R₂)C(═O), N(R₁)(R₂)C(═S) or R₃O—CH₂;

R₁, R₂ and R₃ are independently hydrogen, alkyl or cycloalkyl;

comprising:

contacting a compound of formula (I)

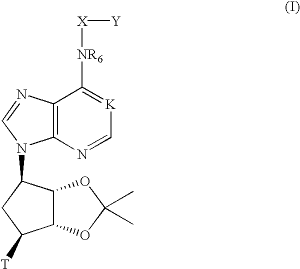

in the presence of an organic solvent, with water containing at least two equivalents of an acid to provide an aqueous medium containing the compound of formula (V) and an organic medium containing organic impurities;

adjusting the pH of the aqueous medium to a basic pH; and

removing the compound of formula (V) from said aqueous medium.

Another embodiment relates to processes for the preparation of a compound of formula (VIII):

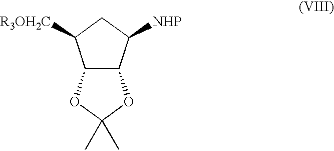

where P is a protecting group and R₃ is alkyl;

comprising protecting a compound of formula (ii):

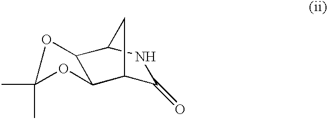

to provide a compound of formula (iii):

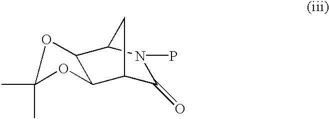

contacting the compound of formula (iii) with a reducing agent to provide a compound of formula (iv):

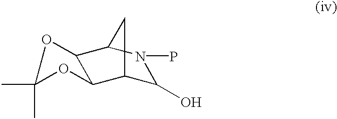

and alkylating the compound of formula (iv) to provide the compound of formula (VIII).

Another embodiment relates to processes for the preparation of a compound of formula (IV):

R₆NH—X—Y (IV)

wherein:

R₆ is hydrogen, alkyl or cycloalkyl;

X is

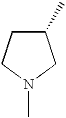

where the nitrogen of the ring of X is substituted by Y;

Y is optionally substituted aryl or optionally substituted heterocyclyl;

comprising contacting a compound of formula (vii):

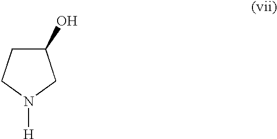

with a compound of formula Y—Z wherein Z is a halogen, in the presence of a first base to provide a compound of formula (ix):

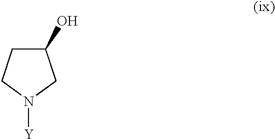

contacting the compound of formula (ix) with a sulfonating agent in the presence of a second base to provide a compound of formula (x):

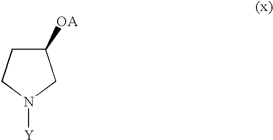

wherein —OA is a sulfonate ester;

contacting the compound of formula (x) with benzylamine to provide a compound of formula (xi):

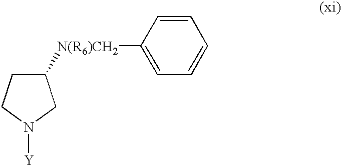

and hydrogenating the compound of formula (xi) in the presence of a hydrogenation catalyst to provide the compound of formula (IV).

Another embodiment relates to intermediates of the foregoing processes. These, as well as other important aspects of the invention will become more apparent from the following detailed description.

### DETAILED DESCRIPTION OF THE INVENTION

As employed above and throughout the disclosure, the following terms, unless otherwise indicated, shall be understood to have the following meanings.

“Acyl” refers to a straight or branched alkyl-C═O group. “Thioacyl” refers to a straight or branched alkyl-C═S group. Preferred acyl and thioacyl groups are lower alkanoyl and lower thioalkanoyl having from 1 to about 6 carbon atoms in the alkyl group, and all combinations and subcombinations of ranges therein.

“Alkyl” refers to a saturated aliphatic hydrocarbon group which may be straight or branched and having from 1 to about 20 carbon atoms in the chain, and all combinations and subcombinations of ranges therein. Preferred alky groups may be straight or branched and have from 1 to about 10 carbon atoms in the chain. Branched means that a lower alkyl group such as, for example, methyl, ethyl or propyl, is attached to a linear alkyl chain.

“Lower alkyl” refers to an alkyl group having from 1 to about 6 carbons, and all combinations and subcombinations of ranges therein.

“Cycloalkyl” refers to an aliphatic ring having from about 3 to about 10 carbon atoms in the ring, and all combinations and subcombinations of ranges therein. Preferred cycloalkyl groups have from about 4 to about 7 carbon atoms in the ring.

“Carbamoyl” refers to an H₂N—C═O group. Alkylcarbamoyl and dialkylcarbamoyl means that the nitrogen of the carbamoyl is substituted by one or two alkyl groups, respectively.

“Carboxyl” refers to a COOH group.

“Alkoxy” refers to an alkyl-O group in which “alkyl” is as previously described. Lower alkoxy groups are preferred. Exemplary alkoxy groups include, for example, methoxy, ethoxy, n-propoxy, i-propoxy and n-butoxy.

“Alkoxyalkyl” refers to an alkyl group, as previously described, substituted by an alkoxy group, as previously described.

“Alkoxycarbonyl refers to an alkoxy-C═O group.

“Aryl” refers to an aromatic carbocyclic radical containing from about 6 to about 10 carbons, and all combinations and subcombinations of ranges therein. Exemplary aryl groups include phenyl and naphthyl.

“Aralkyl” means an alkyl group substituted by an aryl radical. “Optionally substituted aralkyl” and “optionally substituted aryl” means that the aryl group, or the aryl group of the aralkyl group, may be substituted with one or more substituents which include, for example, alkyl, alkoxy, amino, nitro, carboxy, carboalkoxy, cyano, alkyl amino, halo, hydroxy, hydroxyalkyl, mercaptyl, alkylmercaptyl, trihaloalkyl, carboxyalkyl or carbamoyl.

“Aralkoxycarbonyl” refers to an aralkyl-O—C═O group.

“Aryloxycarbonyl” refers to an aryl-O—C═O group.

“Carbalkoxy” refers to a carboxyl substituent esterified with an alcohol of the formula CₙH₂ₙ₊₁OH, wherein n is from 1 to about 6.

“Halogen” (or “halo”) refers to chlorine (chloro), fluorine (fluoro), bromine (bromo) or iodine (iodo). Preferred among the halogens (or halos) is chlorine (or chloro).

“Heterocyclyl” refers to a ring structure containing from about 4 to about 10 members in which one or more of the atoms in the ring is an element other than carbon, e.g., N, O or S. Heterocyclyl groups may be aromatic or non-aromatic, i.e., the rings may be saturated, partially unsaturated, or fully unsaturated. Preferred heterocyclyl groups include, for example, pyridyl, pyridazinyl, pyrimidinyl, isoquinolinyl, quinolinyl, quinazolinyl, imidazolyl, pyrrolyl, furanyl, thienyl, thiazolyl, benzothiazolyl, piperidinyl, pyrrolidinyl, tetrahydrofuranyl, tetrahydropyranyl, and morphonlinyl groups.

“Optionally substituted heterocyclyl” means that the heterocyclyl group may be substituted by one or more substituents wherein the substituents include; for example, alkoxy; alkylamino, aryl, carbalkoxy, carbamoyl, cyano, halo, heterocyclyl, trihalomethyl, hydroxy, mercaptyl, alkylmercaptyl and nitro.

“Hydroxyalkyl” refers to an alkyl group substituted by a hydroxy group. Hydroxy lower alkyl groups are preferred. Exemplary preferred groups include, for example, hydroxymethyl, 2-hydroxyethyl, 2-hydroxypropyl and 3-hydroxypropyl.

“Adenosine compounds” refers to compounds structurally related to adenosine including, for example, adenosine and analogs and derivatives thereof. Preferred adenosine compounds include the compounds disclosed, for example, in WO 98/01426, the disclosures of which are hereby incorporated herein by reference, in their entirety.

“Hydrogenation catalyst” refers to any compounds known in the art of organic synthesis to facilitate the addition of hydrogen. Hydrogenation catalysts include, but are not limited to palladium on carbon, palladium hydroxide on carbon, palladium on calcium carbonate poisoned with lead, and platinum on carbon.

“Sulfonating agent” refers to any reagents known in the art of organic synthesis to react with an alcohol to provide a sulfonate ester. Examples include, but are not limited to methanesulfonyl chloride, methanesulfonic anhydride, trifluoromethane sulfonyl chloride, trifluoromethane sulfonic anhydride, benzene sulfonyl chloride, p-toluenesulfonyl chloride, a p-toluenesulfonyl anhydride. “Sulfonate ester” includes groups which result when a sulfonating agent is reacted with an alcohol in the presence of an acid scavenger to give a compound of form —OA, wherein A is SO₂R′, with R′ deriving from the sulfonating agent.

“Reducing agent” refers to any reagents known in the art of organic synthesis to reduce the oxidation state of a carbon atom, for example, by reducing a ketone to an alcohol. Reducing agents include, but are not limited to hydride derivatives, such as borohydrides, including lithium borohydride and sodium borohydrides.

“Methylating agent” refers to any reagent known in the art of organic synthesis to donate a methyl group to an alcohol to form an ether. Methylating agents include, but are not limited to methylhalides such as methyliodide, methylchloride, methylbromide, and dimethylsulfate.

“Acid scavenger” refers to any species known in the: art of organic synthesis capable of accepting a proton without reacting with the starting material or product.

“Concatenated” refers to multi-step processes (i.e., processes containing two or more steps) wherein the steps may be performed in a substantially continuous or sequential manner, preferably without the necessity for interim isolation and/or purification of the intermediate compounds.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

“Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. Thus, the term “acid addition salt” refers to the corresponding salt derivative of a parent compound which has been prepared by the addition of an acid. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Certain acidic or basic compounds of the present invention may exist as zwitterions. All forms of the compounds, including free acid, free base and zwitterions, are contemplated to be within the scope of the present invention.

The reactions of the synthetic methods described and claimed herein may be carried out in suitable solvents which may be readily selected by one skilled in the art of organic synthesis. Generally, suitable solvents are solvents which are substantially non-reactive with the starting materials (reactants), the intermediates, or products at the temperatures at which the reactions are carried out, i.e., temperatures which may range from the solvent's freezing temperature to the solvent's boiling temperature. A given reaction may be carried out in one solvent or a mixture of more than one solvent. Depending on the particular reaction, suitable solvents for a particular work-up following the reaction may be selected. Suitable solvents, as used herein may include, by way of example and without limitation, chlorinated solvents, hydrocarbon solvents, aromatic solvents, ether solvents, protic solvents, polar aprotic solvents, and mixtures thereof.

Suitable halogenated solvents include, but are not limited to carbon tetrachloride, bromodichloromethane, dibromochloromethane, bromoform, chloroform, bromochloromethane, dibromomethane, butyl chloride, dichloromethane, tetrachloroethylene, trichloroethylene, 1,1,1-trichloroethane, 1,1,2-trichloroethane, 1,1-dichloroethane, 2-chloropropane, hexafluorobenzene, 1,2,4-trichlorobenzene, o-dichlorobenzene, chlorobenzene, fluorobenzene, fluorotrichloromethane, chlorotrifluoromethane, bromotrifluoromethane, carbon tetrafluoride, dichlorofluoromethane, chlorodifluoromethane, trifluoromethane, 1,2-dichlorotetrafluorethane and hexafluoroethane.

Suitable hydrocarbon solvents include, but are not limited to alkane or aromatic solvents such as cyclohexane, pentane, hexane, toluene, cycloheptane, methylcyclohexane, heptane, ethylbenzene, m-, o-, or p-xylene, octane, indane, nonane, benzene, ethylbenzene, and m-, o-, or p-xylene.

Suitable ether solvents include, but are not limited to dimethoxymethane, tetrahydrofuran, 1,3-dioxane, 1,4-dioxane, furan, diethyl ether, ethylene glycol dimethyl ether, ethylene glycol diethyl ether, diethylene glycol dimethyl ether, diethylene glycol diethyl ether, triethylene glycol diisopropyl ether, anisole, or t-butyl methyl ether.

Suitable protic solvents include, but are not limited to water, methanol, ethanol, 2-nitroethanol, 2-fluoroethanol, 2,2,2-trifluoroethanol, ethylene glycol, 1-propanol, 2-propanol, 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, and glycerol.

Suitable aprotic solvents include, but are not limited to dimethylformamide (DMF), dimethylacetamide (DMAC), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, acetonitrile (ACN), dimethylsulfoxide (DMSO), propionitrile, ethyl formate, methyl acetate, hexachloroacetone, acetone, ethyl methyl ketone, ethyl acetate, isopropyl acetate, t-butyl acetate, sulfolane, N,N-dimethylpropionamide, nitromethane, nitrobenzene, and hexamethylphosphoramide.

The present invention is directed to processes for the preparation of adenosine compounds. In certain embodiments, the processes of the present invention include the isolation of synthetic intermediates. In other embodiments, the processes of the present invention are substantially concatenated. The term “substantially concatenated”, as used herein, means that at least about 75% of the process for preparing the adenosine compounds may be concatenated (i.e., at least about 75% of the process steps may be carried out without the necessity of isolating and/or purifying intermediates that may be formed during the process). Preferably, at least about 80% of the process may be concatenated, with at least about 85% concatenation being more preferred. Even more preferably, at least about 90% of the process may be concatenated, with at least about 95% concatenation being more preferred. In particularly preferred embodiments, the present processes may be about 100% concatenated (i.e., completely concatenated).

In connection with the preparation of adenosine compounds, the processes of the present invention may offer improved yields, purity, ease of preparation and/or isolation of intermediates and final product, and more industrially useful reaction conditions and workability over prior art methods of preparation. The present processes are particularly useful for the preparation of adenosine compounds on a large scale, including commercial scale, for example, from multi-kilogram to ton quantities or more of adenosine compound. Specifically, isolation and/or purification steps of intermediates to the adenosine compounds may be advantageously substantially or completely avoided using the processes of the present invention. The present processes may be particularly advantageous in that the adenosine compounds may be obtained in substantially pure form. The term “substantially pure form”, as used herein, means that the adenosine compounds prepared using the present processes may preferably be substantially devoid of organic impurities. The term “organic impurities”, as used herein, refers to organic materials, compounds, etc., other than the desired product, that may be typically associated with synthetic organic chemical transformations including, for example, unreacted starting reagents, unreacted intermediate compounds, and the like. In preferred form, the present processes may provide adenosine compounds that are at least about 75% pure, as measured by standard analytical techniques such as, for example, HPLC. Preferably, the adenosine compounds prepared using the present processes may be at least about 80% pure, with a purity of at least about 85% being more preferred. Even more preferably, the adenosine compounds prepared using the present processes may be at least about 90% pure, with a purity of at least about 95% being more preferred. In particularly preferred embodiments, the adenosine compounds prepared using the present processes may be more than about 95% pure, with a purity of about 100% being especially preferred.

In accordance with a preferred embodiment, the present invention is directed to processes for the preparation of a compound of the formula (I):

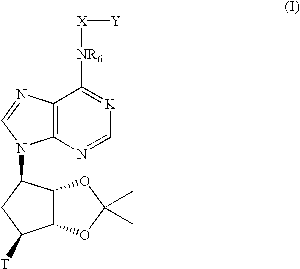

wherein:

K is N, N→O or CH;

R₆ is hydrogen, alkyl, allyl, 2-methylallyl, 2-butenyl or cycloalkyl;

X is

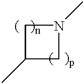

where the nitrogen of the ring of X is substituted by Y;

Y is hydrogen, alkyl, optionally substituted aralkyl, optionally substituted aryl, optionally substituted heterocyclyl or optionally substituted heterocyclylalkyl;

n and p are independently 0, 1, 2 or 3, provided that the sum of n and p is at least 1;

T is hydrogen, alkyl, acyl, thioacyl, halo, carboxyl, N(R₁)(R₂)C(═O), N(R₁)(R₂)C(═S) or R₃O—CH₂;

R₁, R₂ and R₃ are independently hydrogen, alkyl or cycloalkyl;

comprising:

contacting a compound of formula (II)

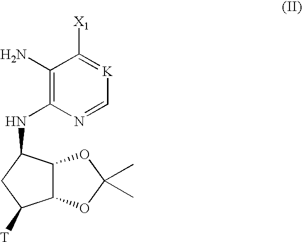

where X₁ is halo; with a formic acid derivative to provide a compound of formula (III):

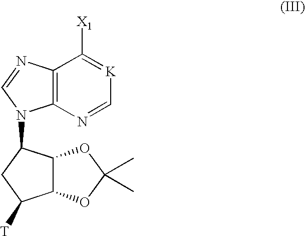

and contacting the compound of formula (III) with a compound of formula (IV)

R₆NH—X—Y (IV)

to provide the compound of formula (I) or a pharmaceutically acceptable salt form thereof. In certain preferred embodiments, the process is substantially concatenated.

In the above process, K is N, N→O or CH. In preferred embodiments, K is N.

R₆ in the above process is hydrogen, alkyl, allyl, 2-methylallyl, 2-butenyl or cycloalkyl. In preferred form, R₆ is hydrogen.

In the above process, X is

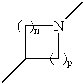

where the nitrogen of the ring of X is substituted by Y. Preferably, X is

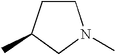

X₁ in the above process is halo. Preferably, X₁ is chloro.

In the above process, Y is hydrogen, alkyl, optionally substituted aralkyl, optionally substituted aryl, optionally substituted heterocyclyl or optionally substituted heterocyclylalkyl. In preferred form, Y is optionally substituted heterocyclyl, with optionally substituted pyridyl being more preferred. Even more preferably, Y is 5-trifluoromethylpyrid-2-yl.

In the process above, n and p are independently 0, 1, 2 or 3, provided that the sum of n and p is at least 1. Preferably, the sum of n and p is 3 or 4.

T in the above process is hydrogen, alkyl, acyl, thioacyl, halo, carboxyl, N(R₁)(R₂)C(═O), N(R₁)(R₂)C(═S) or R₃O—CH₂. In preferred embodiments, T is R₃O—CH₂.

R₁, R₂ and R₃ in the above process are independently hydrogen, alkyl or cycloalkyl. Preferably, R₁, R₂ and R₃ are independently hydrogen or lower alkyl, with hydrogen or methyl being more preferred. Even more preferably, R₁, R₂ and R₃ are methyl.

In the processes for the preparation of compounds of the present compounds, such as, for example, the compounds of formula (I), the compounds of formula (II) may be converted to the compounds of formula (III) using a formic acid derivative. In preferred embodiments, the formic acid derivative is selected from the group consisting of formamidine acetate, an orthoformate ester and dimethylformamide dimethyl acetal. More preferably, the formic acid derivative is formamidine acetate.

In another preferred embodiment of the present invention, there are provided processes for the preparation of compounds of formula (V):

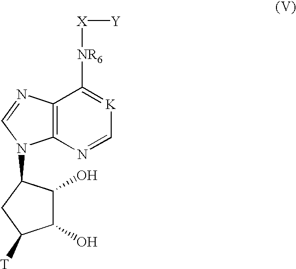

or a pharmaceutically acceptable salt form thereof, wherein K, T, R₆, X and Y are as previously described. Generally speaking, the preparation of compound (V) comprises the deprotection of (I) by contacting the compound of formula (I) with water containing an acid, preferably at least two equivalents of an acid. Suitable acids include, for example, HCl.

In accordance with particularly preferred embodiments, the preparation of the compound of formula (V) comprises contacting a compound of formula (I) in the presence of an organic solvent, with water containing at least two equivalents of an acid to provide an aqueous medium containing the compound of formula (V) and an organic medium containing organic impurities. The preparation of compound (V) further preferably comprises adjusting the pH of the aqueous medium to a basic pH, and removing the compound of formula (V) from the aqueous medium, for example, by extraction with an organic solvent. In especially preferred embodiments, the preparation of (V) further comprises replacing the extraction solvent with a crystallization solvent, and crystallizing the compound of formula (V) from the crystallization solvent. In a further preferred embodiments, the crystallization solvent is selected from the group consisting of acetonitrile, ethyl acetate, methanol, ethanol, isopropanol, butanol, or a combination thereof. In an even more preferred embodiment, crystallizing the compound of formula (V) provides crystals having an average particle diameter which ranges from about 5 to about 50 μm, and all combinations and subcombinations of ranges and specific particle sizes therein. Still more preferably, the methods described herein provide the compound of formula (V) with a particle size of from about 5 to less than about 50 μm, such as about 40 μm, with particle sizes of from about 10 to about 30 μm being yet more preferred. In still more preferred embodiments, the compound of formula (V) is provided with a particle size of from about 15 to about 25 μm, with particle sizes of about 20 μm being especially preferred.

In yet another embodiment of the present invention, there are provided processes for the preparation of a compound of formula (II) comprising:

contacting a compound of formula (VI)

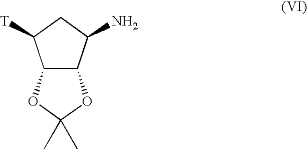

with a compound of formula (VII)

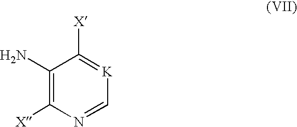

wherein X′ and X″ are independently halo and K and T are as described previously. Preferably, X′ and X″ are chloro.

In yet another embodiment of the invention, there are provided processes for the preparation of the compounds of formula (VI) comprising selectively deprotecting a compound of formula (VIII)

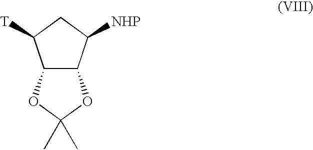

where P is a protecting group and T is described previously. The protecting group P, as well as other protecting groups which may be employed in the present methods, are preferably selected from those which can later be removed selectively. These protecting groups include the following, which are particularly well suited: t-butoxycarbonyl, chloroacetyl, methoxymethyl, trichloro-2,2,2-ethoxycarbonyl, t-butyl, benzyl, p-nitrobenzyl, p-methoxybenzyl, diphenylmethyl, trialkylsilyl, allyloxycarbonyl, and benzyloxycarbonyl groups, wherein the phenyl ring is optionally substituted by halo, alkyl or alkoxy. Among the protecting groups which are particularly well suited are those described in T. W. Greene and P. G. M. Wuts, “Protecting Groups in Organic Synthesis,” Chapter 7, 2nd edition, John Wiley & Sons (1991). The t-butoxycarbonyl (BOC) group is a preferred protecting group.

In yet another embodiment of the present invention, there are provided processes for the preparation of a compound of formula (VIII):

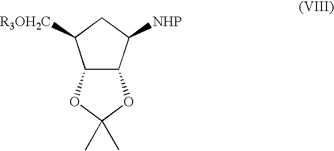

where P is a protecting group and R₃ is alkyl;

comprising protecting a compound of formula (ii):

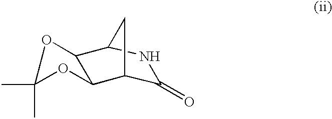

to provide a compound of formula (iii):

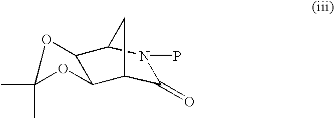

contacting the compound of (iii) with a reducing agent to provide a compound of formula (iv):

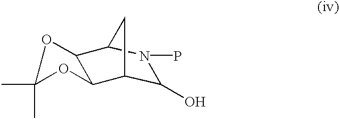

and alkylating the compound of formula (iv) to provide the compound of formula (VIII). In the above process, R₃ is preferably methyl and the protecting group is preferably tert-butyloxycarbonyl. In another preferred embodiment, the reducing agent is selected from the group consisting of lithium borohydride and sodium borohydride. In another preferred embodiment, alkylating comprises contacting the compound of formula (iv) with an alkylating agent selected from the group consisting of CH₃OS(O)₂OCH₃, CH₃I, CH₃Br, CH₃Cl, in the presence of an acid scavenger.

In yet another embodiment of the present invention, there are provided processes for the preparation of a compound of formula (IV):

R₆NH—X—Y (IV)

wherein:

R₆ is hydrogen, alkyl or cycloalkyl;

X is

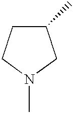

where the nitrogen of the ring of X is substituted by Y;

Y is optionally substituted aryl or optionally substituted heterocyclyl;

comprising contacting a compound of formula (vii):

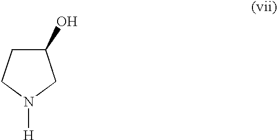

with a compound of formula Y—Z wherein Z is a halogen, in the presence of a first base to provide a compound of formula (ix):

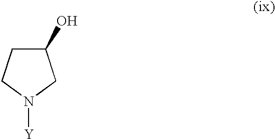

contacting the compound of formula (ix) with a sulfonating agent in the presence of a second base to provide a compound of formula (x):

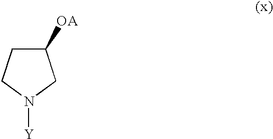

wherein —OA is a sulfonate ester;

contacting the compound of formula (x) with benzylamine to provide a compound of formula (xi):

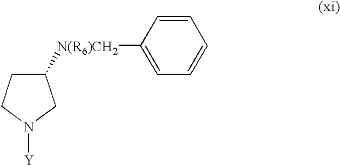

and hydrogenating the compound of formula (xi) in the presence of a hydrogenation catalyst to provide the compound of formula (IV). In the above process, R₆ is preferably hydrogen; Y is preferably

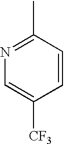

Z is preferably Cl; A is preferably selected from the group consisting of methanesulfonyl, trifluorosulfonyl, p-toluenesulfonyl, and benzenesulfonyl; the first base is preferably selected from the group consisting of Li₂CO₃, K₂CO₃, Na₂CO₃, Cs₂CO₃, NaOH, KOH, and LiOH; the second base is preferably a tertiary amine; and the hydrogenation catalyst is preferably selected from the group consisting of palladium on carbon and palladium hydroxide on carbon.

In yet another embodiment on the present invention, there are provided processes for the preparation of a compound of formula (IV):

R₆NH—X—Y (IV)

wherein:

R₆ is hydrogen, alkyl or cycloalkyl;

X is

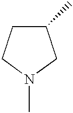

where the nitrogen of the ring of X is substituted by Y;

Y is optionally substituted aryl or optionally substituted heterocyclyl;

comprising contacting a compound of formula (xii):

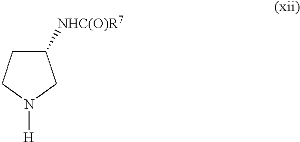

where R⁷ is an optionally substituted alkyl or aryl group,

with a compound of formula Y—Z wherein Z is a halogen, in the presence of a base to provide a compound of formula (xiii):

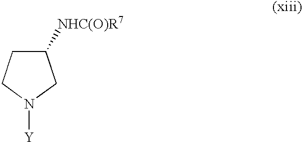

contacting the compound of formula (xiii) with an acid to provide a compound of formula (IV). In the above process R₆ is preferably hydrogen; Y is preferably

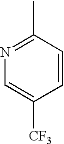

Z is Cl; the base is preferably a tertiary amine; and the acid is preferably selected from the group consisting of hydrochloric acid, hydrobromic acid, sulfuric acid, phosphoric acid, nitric acid, acetic acid, formic acid, trifluoroacetic acid, propionic acid, and methanesulfonic acid.

In yet another embodiment of the present invention, there are provided processes for the preparation of a compound of formula (IV):

R₆NH—X—Y (IV)

wherein:

R₆ is hydrogen, alkyl or cycloalkyl;

X is

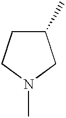

where the nitrogen of the ring of X is substituted by Y;

Y is optionally substituted aryl or optionally substituted heterocyclyl;

comprising contacting a compound of formula (xiv):

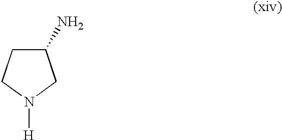

with a compound of formula Y—Z wherein Z is a halogen, in the presence of a base to provide the compound of formula (IV). In the above process, R₆ is preferably hydrogen; Y is preferably

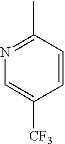

Z is preferably Cl; and the base is preferably selected from the group consisting of Li₂CO₃, K₂CO₃, Na₂CO₃, Cs₂CO₃, NaOH, KOH, and LiOH.

In yet another embodiment of the present invention, there are provided intermediates of the foregoing processes. In a preferred embodiment, the present invention provides a compound of formula (I):

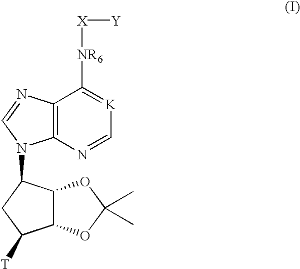

and the salts thereof, wherein:

K is N, N→O or CH;

R₆ is hydrogen, alkyl, allyl, 2-methylallyl, 2-butenyl or cycloalkyl; X is

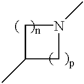

where the nitrogen of the ring of X is substituted by Y;

Y is hydrogen, alkyl, optionally substituted aralkyl, optionally substituted aryl, optionally substituted heterocyclyl or optionally substituted heterocyclylalkyl;

n and p are independently 0, 1, 2 or 3, provided that the sum of n and p is at least 1;

T is hydrogen, alkyl, acyl, thioacyl, halo, carboxyl, N(R₁)(R₂)C(═O), N(R₁)(R₂)C(═S) or R₃O—CH₂; and R₁, R₂ and R₃ are independently hydrogen, alkyl or cycloalkyl.

In a preferred embodiment of the compound of formula (I), the compound is an acid addition salt. In particularly preferred embodiment, the acid addition salt is the hydrochloric acid or methanesulfonic acid salt.

In yet another embodiment of the present invention, there are provided compounds of formula (VI):

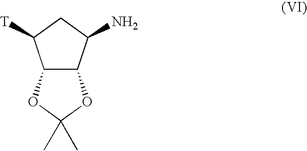

and the salts thereof; wherein T is hydrogen, alkyl, acyl, thioacyl, halo, carboxyl, N(R₁)(R₂)C(═O), N(R₁)(R₂)C(═S) or R₃O—CH₂. In a preferred embodiment, the compound is an acid addition salt. In a particularly preferred embodiment, the salt is derived from hydrochloric acid, oxalic acid, or methanesulfonic acid.

The processes of the present invention, by way of example and without limitation, may be further understood by reference to Schemes 1, 2, and 3.

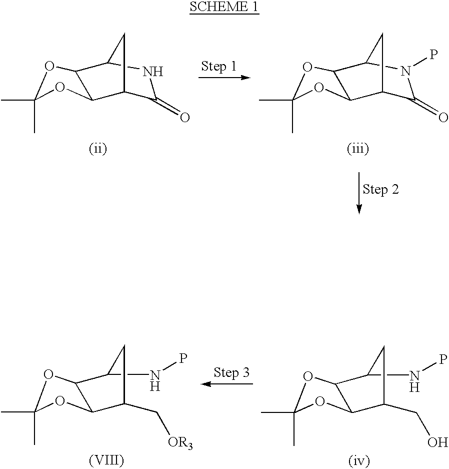

The protected carbosugar (VIII) may be prepared using the methods set forth in Scheme 1 as well as other methods within the scope of the present invention. In Step 1, lactam (ii) is preferably protected with a suitable protecting group (P) to provide compound (iii). Protecting groups and conditions for their use will be readily apparent to one of ordinary skill in the art and include those described in T. W. Greene and P. G. M. Wuts, “Protecting Groups in Organic Synthesis,” Chapter 7, 2nd edition, John Wiley & Sons (1991).

By way of general guidance, solid lactam (ii) may be charged to a reactor along with an excess of suitable acid scavenger. Suitable acid scavengers include, but are not limited to amines, with secondary amines and tertiary amines being preferred. Exemplary amines for use as acid scavengers include, for example, 4-dimethylaminopyridine, triethylamine, diisoproylethylamine, N-methylmorpholine and pyridine, with 4-dimethylaminopyridine being preferred. The vessel may then be charged with a suitable solvent accompanied by moderate heating. Although numerous solvents are possible, aromatic solvents, such as toluene, are preferred. A derivative of a suitable protecting group may then be added to the reaction mixture. In certain preferred embodiments, the protecting group is tert-butyldicarbonate. Thus, a slight molar excess of di-tert-butyldicarbonate may be added to the mixture to provide (iii-i). Upon completion of the reaction (about 2 hours), the mixture may be used directly in the next step.

To perform Step 2, the process stream from Step 1 may be charged to a separate vessel and cooled to about 5 to about 20° C. with moderate stirring. The reactor is preferably charged with a suitable reducing agent, the choice of which will be readily apparent to one of ordinary skill in the art. Preferably, the reducing agent is a hydride, such as lithium or sodium borohydride. The reducing agent is preferably used in a slight excess, i.e., about 1.1 mole, based on compound (iii). The reducing agent is typically dissolved in an appropriate solvent such as methanol, ethanol, propanol, isopropanol, tetrahydrofuran, and the like, prior to addition to the vessel. The mixture may be heated slightly once the exotherm has subsided. After completion of the reaction (ca. 24 h), the mixture is washed with a mild acid and water, the aqueous layers combined, and back extracted with reaction solvent. The organic layers may be combined and used directly in the next step without further work-up or purification.

To perform Step 3, the process stream from Step 2 and about 50% aqueous base, such as hydroxide may be charged to a reaction vessel. The aqueous base is preferably in an excess, for example, a 2 to 10 fold excess based on compound (iv). More preferably, the base is in a 4 to 6 fold excess. The solution is preferably cooled to about 5 to about 20° C. while stirring is maintained at a moderate speed. The reactor may then be charged with an alkylating agent, such as an alkylhalide or dialkylsulfate, dropwise over about 15 to about 60 minutes. The alkylating agent is preferably used in an excess, for example, about 1.4 to about 1.8 mole based on compound (iv). After the exotherm subsides, the mixture may be mildly heated, for example, to about 20 to about 25° C. After completion of the reaction (ca. 2 h), the mixture may be treated with a suitable base, such as ammonium hydroxide, and allowed to react for an additional period of time. The layers may then be split and the aqueous phase discarded. The organic layer may then be washed with water and diluted with a higher boiling solvent for distillation purposes. For example, if toluene is used as the reaction solvent, propylene glycol is a preferred higher boiling solvent. After the distillation is completed, the resultant solution may be treated with water from which the product preferably crystallizes. The solids may then be collected by filtration and washed with water to provide the alkylated product (VIII).

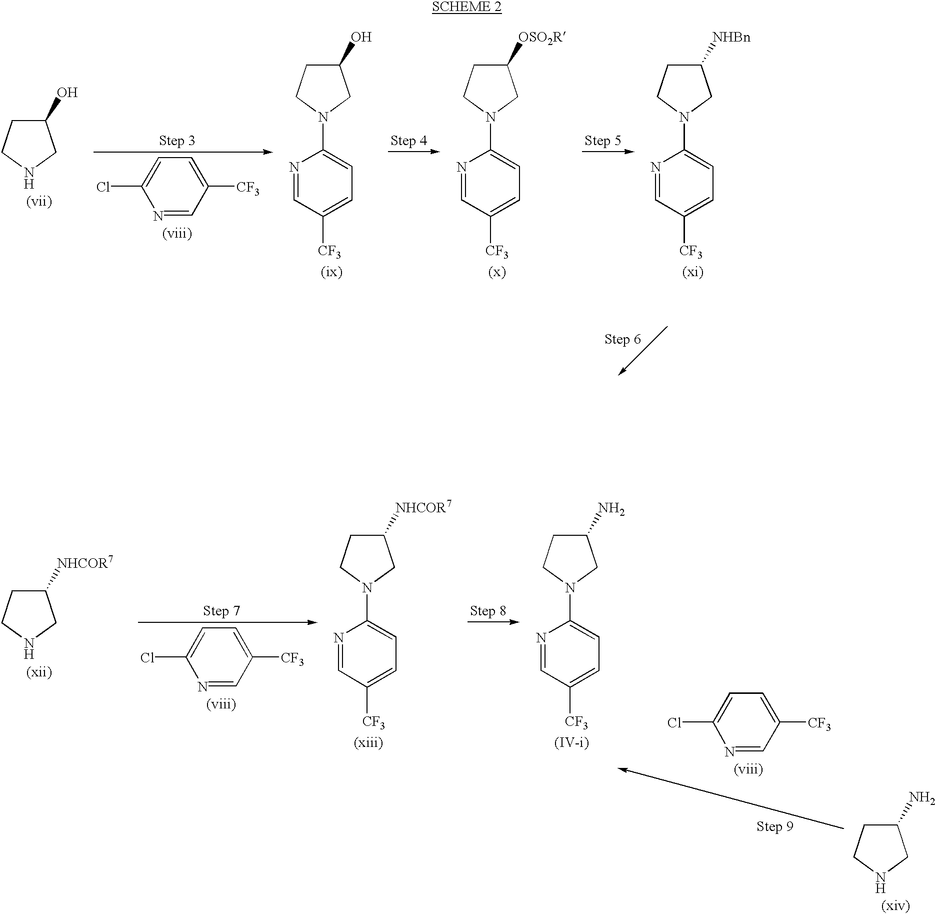

The compound 1-(5-trifluoromethyl-pyridin-2-yl)-pyrrolidin-3(S)-ylamine (IV-i) may be prepared using the methods set forth in Scheme 2 as well as other methods within the scope of the present invention.

In Step 3, 2-chloro-5-trifluoromethyl-pyridine (viii) and 3(R)-hydroxypyrrolidine hydrochloride (vii), preferably in approximately equal molar amounts, may be charged to a vessel containing a suitable solvent. In certain embodiments, water may also added to the vessel. Although numerous solvents are possible, DMSO or alcohol solvents, such as methanol, ethanol, isopropanol, and the like, are preferred. A base, preferably a carbonate, including, but not limited to lithium, sodium, potassium, and cesium carbonate is charged to the vessel. Potassium carbonate is more preferred. In a particularly preferred embodiment, DMSO is charged to the reaction vessel and heated, followed by the addition of compound (vii), the base, and melted (viii). In any case, the reaction mixture is preferably heated for several hours, after which ion pair chromatography may be used to monitor the consumption of starting material. By way of general guidance, the reaction may be heated to about 50 to about 100° C., depending upon the solvent used. Upon completion, water may be charged to the reaction vessel while maintaining the elevated temperature for about 1 hour. The reaction is preferably cooled and the product isolated by filtration.

In Step 4, compound 1-(5-trifluoromethyl-pyridin-2-yl)-pyrrolidin-3(R)-ol (ix) and a suitable solvent may be charged to a reactor with stirring. An excess of acid scavenger is preferably added. Suitable acid scavengers include, but are not limited to amines, with secondary amines and tertiary amines being more preferred. Exemplary amines include, for example, 4-dimethylaminopyridine, triethylamine, diisoproylethylamine, N-methylmorpholine and pyridine, with triethylamine being preferred. The addition of the acid is followed by the addition of an reagent capable of forming a leaving group upon reaction with the hydroxy group of compound (ix). Such reagents are well-known in the art of organic synthesis and include, but are not limited to sulfonyl chlorides. Thus, suitable reagents for this purpose include, but are not limited to alkyl sulfonyl chlorides such as methyl, ethyl, and the like, and aromatic sulfonyl chlorides, such as benzene and toluyl. Methane sulfonyl chloride is more preferred. The reagent may be added over a period of time so as to control the internal temperature below about 20° C. After addition is complete, the internal temperature will typically fall with additional stirring. The mixture may then be warmed, during which time a solid precipitate (amine HCl) may be observed.

The reaction may be monitored by TLC or LC. Upon completion, the reaction mixture may be washed with aqueous base and the aqueous layer back-extracted with a suitable organic solvent. The combined organic extracts are preferably washed with brine and re-charged to the reactor. The organic solvent may then be distilled accordingly and an additional solvent added to provide a solvent system suitable for crystallization. By way of general guidance, the aqueous layer may be extracted with methylene chloride (CH₂Cl₂) and isopropanol (IPA) added during distillation to provide about a 80/20 IPA/CH₂Cl₂ system. Typically, solids precipitate in the reactor, at which point the mixture is preferably cooled to ambient temperature. The mixture may then be cooled and filtered to provide compound (x).

In Step 5, benzylamine and an excess of compound (x) may be charged to a reaction vessel. The reaction mixture is preferably warmed to over about 50° C., more preferably over about 100° C., to form a clear solution and held for a period of time such that HPLC analysis shows negligible starting material. The mixture is preferably cooled, followed by the addition of a suitable solvent for work-up. Although numerous solvents are possible, ether solvents, such as methyl-tert-butyl ether are preferred. The organics may be washed with aqueous base, brine, and diluted with additional solvent. Carbon dioxide is preferably bubbled into the solution to form a precipitate which may be removed by filtration. The filtrate may be transferred to a distillation vessel and distilled at atmospheric pressure over several hours. If desired, the remaining solution may be replaced during distillation with a solvent suitable for the next reaction sequence. Alcohol solvents are preferred, with ethanol being even more preferred.

In Step 6, compound (xi) may be taken up in an alcohol solvent, preferably methanol or ethanol. In certain embodiments, an additional solvent may be added, such as an ether solvent, with methyl-tert-butyl ether being preferred. An acid may be added to catalyze the reaction. Although numerous acids may be used, mineral acids, including hydrochloric acid, and organic acids, including glacial acetic acid are preferred. Glacial acetic acid is even more preferred. Following the addition of the acid, the mixture may be stirred which is typically accompanied by a slight elevation in temperature. A slurry of hydrogenation catalyst may then be added. Hydrogenation catalysts are well-known in the art and include, but are not limited to palladium derivatives. A particularly preferred palladium catalyst is 5% Pd/C. Preferably, the catalyst will be in a slurry of a suitable solvent, such as methanol or ethanol.

The mixture may be warmed followed by the addition of ammonium formate. Depending upon the progression of the reaction, which may be monitored by ion pair chromatography, additional acetic acid and ammonium formate may be desired. Upon reaction completion, the catalyst may be removed by filtration through, for example, celite, glass microfibre paper, or the like. The solids are preferably washed with alcohol solvent. Care should be taken to keep the catalyst bed wet. The combined organics may be distilled to a minimum volume in which stirring can be maintained. An addition solvent suitable for azeotropic distillation of the alcohol solvent may be added. The use of solvents for azeotropic distillation is well-understood in the art. By way of guidance, if ethanol is used during hydrogenation, ethyl acetate may be added and the mixture distilled azeotropically to remove, for example, EtOH/EtOAc. Once the solvent removal is deemed sufficient, additional solvent and water may be added for work-up. The mixture is preferably cooled to below about 25° C., which typically results in phase separation. The pH of the aqueous phase may be adjusted to a basic, for example, below pH 9, more preferably, below pH 10 with an aqueous base such as hydroxide. The layers may then be separated and the aqueous layer extracted with a suitable solvent.

The organic layers may be combined and an acid added to provide an acid addition salt. Such acid addition salts are well-known in the art and include, but are not limited to the salts described herein. Preferred acid addition salts of compound (IV) are formed via the addition of (+)-mandelic acid or methane sulfonic acid. By way of general guidance, the acid may be directly added to an organic solution of the product. After several minutes, a solid preferably precipitates. The mixture may be heated to cause dissolution of the precipitate, followed by distillation to collect an amount of distillate suitable to reprecipitate the product. Once precipitation has occurred, additional solvent may be added to the mixture accompanied by cooling. The solids may then be collected by filtration, washed with additional solvent, and dried to form the desired salt.

An alternate preparation of (IV-i) is provided by Steps 7 and 8 in Scheme 2. As will be recognized, C(O)R⁷ of compound (xii) may be various acyl groups including, but not limited to compounds wherein R⁷ is optionally substituted alkyl or aryl. By way of general guidance, 3S-acetamidopyrrolidine (xii-i; R″ is methyl) and 2-chloro-5-trifluoromethyl pyridine (viii) may be melted in a hot water bath, for example, at a temperature of about 75 to about 85° C., and poured into a suitable reaction vessel. The mixture is preferably diluted with a suitable solvent followed by the addition of an 5 excess of a suitable base. Although numerous solvents are possible, alcohol solvents are preferred, with ethanol being more preferred. Suitable bases include, but are not limited to tertiary amine bases. Tertiary trialkyl amine bases are particularly preferred. The base may be added to the stirred mixture in a steady stream over, for example, about 15 to about 30 minutes. The stirred mixture is preferably heated at reflux for about 15 to about 48 hours and the reaction monitored by process control analysis (HPLC). Once the reaction has reached completion, the mixture may be cooled then quenched with an acid that is preferably introduced into the reaction vessel by slow addition. Although numerous acids are possible, mineral acids are preferred,-with hydrochloric acid being more preferred. The mixture may be heated to boiling and solvent distilled as needed. The mixture may be heated at reflux additional period of time to effect hydrolysis.

In process control analysis may also be used to monitor the presence of starting material (xiii). Once the reaction is deemed complete, the mixture may be quenched by pouring the contents of the reaction vessel into water. The acid may be neutralized by the addition of an aqueous base, such as hydroxide, at reduced temperature. Typically, the product precipitates and a suitable extraction solvent, such as an acetate, may be added followed by additional aqueous base. The phases may be split to provide the organic phase. Additional solvent accompanied by agitation may be necessary to achieve separation of the layers as will be appreciate by one of ordinary skill in the art.

The combined organic extracts may be washed with saturated aqueous sodium chloride solution and heated to distill, for example, excess organic base and/or water. If an acid addition salt is desired, the mixture may be treated dropwise with a suitable acid. As noted above, acid addition salts are well-known in the art and include, but are not limited to the salts described herein. Typically, crystals form immediately upon the initiation of the acid addition. The mixture may be filtered and washed with a suitable solvent to provide the desired salt.

An alternate preparation of (IV-i) from 2-chloro-5-trifluoromethylpyridine (xiv) and 2-S-aminopyrrolidine (viii) is provided by Step 9 in Scheme 2. By way of general guidance, 2-chloro-5-trifluoromethylpyridine, 2-S-aminopyrrolidine, an excess of a suitable base, and solvent may be charged to a reaction vessel. Although numerous bases are possible, carbonates are preferred, with potassium carbonate being more preferred. Suitable solvents will be readily apparent to one of ordinary skill and may be effected by the choice of base. For example, if a carbonate is used, preferable solvents include, but are not limited to alcohol solvents, such as methanol and ethanol. The mixture is preferably heated until the reaction is deemed complete by HPLC. By way of general guidance, if the reaction is heated at about 70 to about 80° C., the reaction should be complete after about 20 hours. The reaction is then preferably filtered hot. The filtrate may be cooled to room temperature, followed by the addition of a suitable acid. A solid typically forms immediately. The solid may be filtered and dried to give the desired salt.

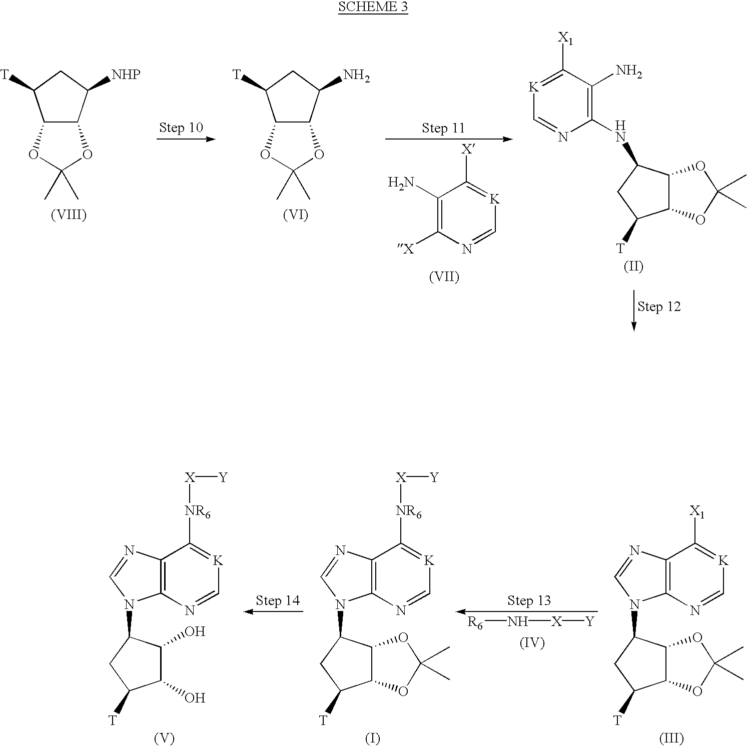

In Step 10, a suitable compound of formula (VIII) may be combined with about 5 liters of a suitable solvent per kilogram of compound (VIII), preferably under an inert atmosphere. A suitable acid may be added to selectively deprotect the amine portion of the compound. While a wide range of solvents may be used, such as halogenated, protic, aprotic, hydrocarbon or ethers, polar aprotic solvents may be preferred. Exemplary polar solvents include, for example, ethyl acetate (EtOAc), propyl acetate (PrOAc) and butyl acetate (BuOAc), with ethyl acetate and butyl acetate being preferred. Other solvents that may be suitable for use in Step 10 would be readily apparent to one of ordinary skill in the art, once armed with the teachings of the present application.

Suitable acids for use in Step 10 include, for example, mineral and organic acids. Preferred among these acids are HCl, oxalic acid, D-tartaric acid, L-tartaric-acid, succinic acid, fumaric acid, formic acid, benzoic acid, dibenzoyl-D-tartaric acid, dibenzoyl-L-tartaric acid, di-p-toluoyl-D-tartaric acid, di-p-toluoyl-L-tartaric acid. More preferred is hydrochloric acid and oxalic acid. As would be readily apparent to one of ordinary skill in the art, deprotection of compound (VIII) may result in the formation of an amine salt. Thus, the choice of acid may depend upon the particular salt desired. Particularly preferred salts are the HCl salt and the oxalate salt. Other preferred salts would be apparent once armed with the teachings of the present application.

The quantity of acid employed to deprotect the compound of formula (VIII) may vary depending, for example, on the particular compound (VIII) and the particular acid employed. Generally speaking the quantity of acid employed may range from about 2 to about 8 equivalents, and all combinations and subcombinations of ranges therein, based on the compound (VIII). More preferably, about 3 to about 6 equivalents of acid may be employed, with about 4.5 equivalents being even more preferred.

By way of general guidance, the deprotection of compound (VIII) may be conducted over a wide range of temperatures. Preferably, the reaction is conducted at a temperature and for a time sufficient to form the compound of formula (VI). The particular temperatures and times may vary, depending, for example, on the particular compound (VIII) and acid involved, as well as the particular solvent employed. In preferred form, the mixture of compound (VIII) and solvent may be cooled prior to the acid addition, preferably to a temperature of from about 0 to about 15° C., with a temperature of about 10° C. being more preferred. The acid may then be added to the cooled mixture, and the reaction temperature may be permitted to warm, for example to room temperature, during the addition of the acid. The reaction may be continued for a suitable period of time, for example, from about 0.5 to about 5 hours, preferably for about 1 hour. The reaction may be monitored by standard analytical techniques, such as thin layer chromatography (TLC). After the reaction in Step 10 has reached the desired state of completion, the reaction mixture may be advantageously employed in Step 11 as is, i.e., without further purification-and/or isolation of the compound of formula (VIII).

In Step 11, a suitable compound of formula (VII) and a suitable acid may be added to the crude reaction mixture from Step 10. Alternatively, if the compound from Step 10 is isolated, it may be dissolved in a suitable solvent and added to a suitable compound of formula (VII), which may be dissolved in the same or different suitable solvent, preferably containing a suitable base. A wide variety of compounds of formula (VII) are commercially available and/or may be prepared using standard synthetic organic chemistry techniques. The suitable base and the amount thereof, should be judiciously chosen so it may serve both as an acid scavenger and as an agent to free base the compound of formula (VI), if necessary. As will be recognized, a wide variety of acid scavengers may be employed in Step 11 for these purposes. Preferred acid scavengers are amines, with secondary amines and tertiary amines being more preferred. Exemplary amines include, for example, triethylamine, diisoproylethylamine, N-methylmorpholine and pyridine, with diisoproylethylamine being preferred. Other preferred acid scavengers include, for example, carbonates and bicarbonates. Other scavengers that may be suitable for use in Step 11 would be readily apparent to one of ordinary skill in the art, once armed with the teachings of the present application.

The quantity of acid scavenger employed may vary depending, for example, on the particular acid scavenger and compounds (VI) and (VII) involved in Step 11. Generally speaking the quantity of acid scavenger employed may range from about I to about 8 equivalents, and all combinations and subcombinations of ranges therein, based on compound (VI). More preferably, about 2 to about 5 equivalents of acid scavenger may be employed, with about 3.5 equivalents being even more preferred. The quantity of the compound of formula (VII) employed may also vary depending, for example, on the particular compounds (VI) and (VII) involved in the reaction. Generally speaking, a molar excess of compound (VII) may be employed, for example, from about 5 to about 10% excess, based on compound (VI).

By way of general guidance, the reaction of compound (VI) with compound (VII) may be conducted over a wide range of temperatures. Preferably, the reaction is conducted at a temperature and for a time sufficient to form the compound of formula (II). As will be recognized, it may be advantageous to add compounds (VII) and (VIII) at a lower temperature, followed by temperature, ramping to a preferred temperature for forming, compound (II): The particular temperatures and times may vary, depending, for example, on the particular compounds (VII) and (VIII) involved, as well as the particular solvent employed. In preferred embodiments, the reaction of compound (VI) with compound (VII) may be conducted at an elevated temperature, preferably at a temperature of from about 50° C. to about 150° C., and all combinations and subcombinations of ranges therein. More preferably, the reaction of compound (VI) with compound (VII) may be conducted at a temperature of from about 75° C. to about 140° C., with temperatures of about 100° C. to about 130° C. being even more preferred. In a particularly preferred embodiment, the reaction of compound (VI) with compound (VII) may be conducted at a temperature of about 125° C. In an alternative particularly preferred embodiment, the reaction of compound (VI) with compound (VII) may be conducted at a temperature of about 105° C. Preferred reaction times may range from about 5 to about 120 hours, and all combinations and subcombinations of ranges therein. The reaction progress may be monitored by standard analytical techniques, such as high pressure liquid chromatography (HPLC).

As with Step 10, a wide range of solvents may be used in Step 11, such as halogenated, protic, aprotic, hydrocarbon or ethers. Preferred solvents include polar solvents including, for example, water (H₂0), ethyl acetate (EtOAc), propyl acetate (PrOAc), butyl acetate (BuOAc), dimethylsulfoxide (DMSO) and 1-methyl-2-pyrrolidinone (NMP). As noted above, Step 11 may preferably be conducted at elevated temperatures. In accordance therewith, the solvent employed in Step 11 is preferably selected from the higher boiling of the aforementioned solvents, preferably H₂O, BuOAc, DMSO or NMP. Thus, if Step 10 is conducted in a lower boiling solvent, such as, for example, EtOAc, the lower boiling solvent may be preferably exchanged in Step 11, for example, via distillation, for a higher boiling solvent.

By way of example and without limitation, Step 10 may be conducted, for example, in EtOAc. After Step 10, the EtOAc may be distilled off, and the remaining crude reaction mixture may be dissolved or suspended in a higher boiling solvent, such as BuOAc or DMSO, for further reaction in Step 11. While either of these solvents may be suitable for use in Step 11, it has been surprisingly and unexpectedly discovered that DMSO may be particularly advantageous in that it may provide substantial improvement in the rate of the reaction of compound (VI) with compound (VII). Moreover, once the reaction of Step 11 has reached the desired stage of completion, the DMSO may be easily exchanged for a water-immiscible organic solvent by diluting the DMSO reaction mixture with water and extracting the diluted reaction mixture with a solvent of interest, for example, BuOAc.

After the reaction in Step 11 has reached the desired state of completion, the reaction mixture may be cooled. If desired, the crude reaction mixture may be washed with water and/or a dilute acid to remove any impurities and unreacted starting materials. A wide variety of acids may be used in the acid wash, including mineral and organic acids, with organic acids being preferred. Preferred among the organic acids is citric acid. The resultant compound may be isolated and purified or, alternatively, the crude reaction mixture may then be used as is (i.e.,without further isolation and/or purification) in the next reaction step.

In Step 12, a suitable formic acid derivative may be added to the crude reaction mixture from Step 11. A wide variety of formic acid derivatives may be employed in Step 12, including, for example, formamidine acetate, an orthoformate ester and dimethylformamide dimethyl acetal. Preferred among the formic acid derivatives is formamidine acetate.

It may be advantageous to include a catalyst, preferably an acid catalyst, in Step 12. The acid catalyst may be selected, for example, from mineral acids and organic acids. Preferred acid catalysts include, for example, trifluoroacetic acid, acetic acid, and formic acid and salts thereof, such as ammonium formate. The use of a catalyst may depend, for example, on the particular formic acid derivative employed. For example, formamidine acetate (HC(═NH)NH₂.CH₃CO₂H) includes an acid moiety (i.e., acetic acid) which, as noted above, may serve as a catalyst. Other formic acid derivatives, such as orthoformate esters and dimethylformamide dimethyl acetal, do not include such acidic moieties, and a catalyst is preferably used with such formic acid derivatives.

The quantity of formic acid derivative employed may vary depending, for example, on the particular compound (II) and formic acid derivative involved in the reaction. Generally speaking, the formic acid derivative may be employed in excess, preferably ranging from about 2 to about 8 equivalents, and all combinations and subcombinations of ranges therein, based on the compound (II). More preferably, from about 3 to about 6 equivalents of formic acid derivative may be employed, with about 4 equivalents being even more preferred.

By way of general guidance, the reaction of compound (II) with the formic acid derivative may be conducted over a wide range of temperatures. Preferably, the reaction is conducted at a temperature and for a time sufficient to form the compound of formula (III). The particular temperatures and times may vary, depending, for example, on the particular compound (II) and formic acid derivative involved, as well as the particular solvent employed. In preferred embodiments, the reaction of compound (VI) with compound (VII) may be conducted at an elevated temperature, preferably at a temperature of from about 50° C. to about 150° C., and all combinations and subcombinations of ranges therein. In preferred form, the reaction of compound (II) with the formic acid derivative may be conducted temperature of about 100° C. to about 140° C., with temperatures of about 120° C. to about 130° C. being even more preferred. In particularly preferred embodiments, the reaction of compound (II) with a formic acid derivative may be conducted at a temperature of about 125° C. Preferred reaction times may range from about 5 to about 120 hours, and all combinations and subcombinations of ranges therein. The reaction progress may be monitored by standard analytical techniques, such as HPLC.

A wide range of solvents may be used in Step 12, such as halogenated, protic, aprotic, hydrocarbon or ethers. Preferred solvents are polar solvents including, for example, EtOAc, PrOAc and BuOAc, DMSO and NMP. Accordingly, the solvent employed in Step 12 is preferably selected from among the higher boiling of the aforementioned solvents, preferably BuOAc, DMSO and NMP. BuOAc is particularly preferred for use in Step 12.

After the reaction in Step 12 has reached the desired state of completion, the reaction mixture may be cooled, for example, to room temperature. If desired, the crude reaction mixture may be washed with water and/or a dilute acid to remove any impurities and unreacted starting materials. Suitable acids for use in the acid wash include, but are not limited to the acids discussed above in connection with Step 11. Distillative processes may then be used to remove residual water, as well as excess reaction solvent. In preferred embodiments, from about 10% to about 50% of the original solvent volume may be removed by distillation, with from about 20% to about 40% being preferred. In particularly preferred embodiments, about 35% of the original solvent volume may be removed by distillation. As will be appreciated by one of ordinary skill in the art, additional solvents may be added during the distillation process to drive off the previous solvent and to obtain the reaction product in the solvent to be used in the next reaction. Thus, preferred replacement solvents will be those contemplated for use in the proceeding reaction. Particularly preferred solvents for use in Step 13 include, but are not limited to DMSO and n-BuOAc. More preferred is DMSO. The crude reaction mixture may then be used as is (i.e.,without further isolation and/or purification) in the next reaction step.

In Step 13, a suitable compound of formula (IV) may be added to the reaction mixture from Step 12. Preferred compounds of formula (IV), and methods for their preparation, are discussed in detail below. The quantity of compound (IV) employed may vary depending, for example, on the particular compounds (III) and (IV) involved in the reaction. Generally speaking, compound (IV) may be employed such that there is a molar excess of compound (III), for example, from about 5 to about 10% excess of (III).

In preferred embodiments, a suitable acid scavenger may also be included in the reaction in Step 13. In certain preferred embodiments, the acid scavenger is in solution with the compound of formula (IV) prior to the addition of the compound of formula (III). Among the suitable acid scavengers are those described above in connection with Step 11, including amine bases wherein triethylamine and diisopropylethylamine are preferred. Diisopropylethyl amine is more preferred. The quantity of acid scavenger employed may vary depending, for example, on the particular compounds (III) and (IV) and acid scavenger employed. Generally speaking the quantity of acid scavenger employed may range from about 1 to about 8 equivalents, and all combinations and subcombinations of ranges therein, based on compound (III). More preferably, from about 2 to about 5 equivalents of acid scavenger may be employed, with about 3.5 equivalents being even more preferred.

By way of general guidance, the reaction of compound (III) with compound (IV) may be conducted over a wide range of temperatures. Preferably, the reaction is conducted at a temperature and for a time sufficient to form the compound of formula (I). The particular temperatures and times may vary, depending, for example, on the particular compounds (III) and (IV) involved, as well as the particular solvent employed. In preferred embodiments, the reaction of compound (III) with compound (IV) may be conducted at an elevated temperature, preferably at a temperature of from about 35° C. to about 120° C., and all combinations and subcombinations of ranges therein. In preferred form, the reaction of compound (III) with (IV) may be conducted at a temperature of about 50° C. to about 100° C., with temperatures of about 75° C. to about 90° C. being more preferred. In particularly preferred embodiments, the reaction of compound (III) with (IV) may be conducted at a temperature of about 85° C. Preferred reaction times may range from about 4 to about 48 hours, and all combinations and subcombinations of ranges therein. The reaction progress may be monitored by standard analytical techniques, such as HPLC.

A wide range of solvents may be used in Step 13, such as halogenated, protic, aprotic, hydrocarbon or ethers or mixtures thereof including, for example, mixtures of a hydrocarbon solvent (such as, for example, toluene) and a protic solvent (such as, for example, water). Preferred solvents are polar solvents including, for example, EtOAc, PrOAc and BuOAc, DMSO and NMP. BuOAc and DMSO are particularly preferred for use in Step 13. In certain preferred embodiments, a minor amount of a protic solvent may also be included in the reaction of Step 13. Suitable protic solvents include, for example, methanol, ethanol and isopropanol, with ethanol being preferred. The amount of protic solvent employed may vary depending, for example, on the particular reagents and reaction solvent employed.

After the reaction in Step 13 has reached the desired level of completion, the crude reaction mixture may then be advantageously employed in Step 14 as is, i.e., without further purification and/or isolation of the compound of formula (I). Alternatively, the reaction may be partitioned between a suitable solvent and water to remove impurities by washing as described in connection with Step 12. Suitable solvents for this purpose will be readily apparent to one of ordinary skill in the art, however, polar solvents are preferred. Butyl acetate is particularly preferred. Following washing, the organic phase may be azeotropically dried and concentrated.

Alternatively, if a salt of the compound of formula (I) is desired, a suitable acid may be added followed by cooling and seeding of the resultant solution to provide the crystalline salt. Preferably, the acid chosen will be able to form the salt without effecting the integrity of the target compound. Thus, mild acids, such as sulfonic acids, are preferred. In particular, methane sulfonic acid, benzenesulfonic acid, toluenesulfonic acid, hydroxyethanesulfonic acid, camphorsulfonic acid, and other sulfonic acids may prepare suitable crystalline salts. A particularly preferred acid is methane sulfonic acid. It will be appreciated, however, that numerous other salts are possible, when an anhydrous form of the acid is available. For example, mineral acids, such as hydrochloric, hydrobromic, phosphoric, sulfuric, or nitric acid may prepare suitable crystalline salts. Other organic acids, such as fumaric, succinic, oxalic, citric, and the like, may prepare suitable crystalline salts provided that they are sufficiently acidic to protonate the basic moiety of compound (I). As noted above, butyl acetate is a preferred solvent when, for example, the reaction is partitioned between a suitable solvent and water to remove impurities by washing as described in connection with Step 12. Advantageously, butyl acetate is also preferred for isolation of the salts of the compound of formula (I).

Under appropriate conditions, however, other solvents may be used to prepare crystalline salts of (I), such as ester solvents, including, but not limited to ethyl acetate, propyl acetate, isopropyl acetate, isobutyl acetate, ethyl propionate, propyl propionate, isopropyl propionate; ether solvents, including, but not limited to t-butyl methyl ether, tetrahydrofuran, ethyl ether, isopropyl ether, butyl ether; and aromatic solvents, including, but not limited to toluene and anisole. Other solvents will be readily understood to those of ordinary skill in the art. Filtration and washing of the product, preferably with additional crystallization solvent, affords the compound of formula (I).

In Step. 14, the compound of formula (I) may be deprotected with a suitable acid to form the compound of formula (VI). Suitable acids include, for example, mineral and organic acids, with mineral acids being preferred. Suitable acids include, for example, HCl, HBr, H₂SO₄, HNO₃ and acetic acid, with HCl being preferred. The quantity of acid employed to deprotect the compound of formula (I) may vary depending, for example, on the particular acid employed, and the particular compound (I) involved. Generally speaking, a molar excess of acid may be employed, with at least 2 equivalents of acid being preferred. More preferably, from about 2 to about 5 equivalents of acid may be employed, with from about 2 to about 3 equivalents being even more preferred.

By way of general guidance, the deprotection of compound (I) may be conducted over a wide range of temperatures. Preferably, the reaction is conducted at a temperature and for a time sufficient to form the compound of formula (V). The particular temperatures and times may vary, depending, for example, on the particular compound (I) and acid involved, as well as the particular solvent employed. In preferred form, the deprotection of compound (I) may be conducted at a temperature of from about 10 to about 35° C., and all combinations and subcombinations of temperature ranges therein. More preferably, the deprotection of compound (I) may be conducted at about room temperature.

Generally speaking, the deprotection in Step 14 may be conducted by contacting the reaction mixture from Step 13 with the aqueous acid solution. It has been surprisingly and unexpectedly discovered that, as the reaction proceeds, impurities and unreacted compound (I) remain in the organic solvent layer. This enables facile and convenient isolation of the compound of formula (I). In certain embodiments, the compound of formula (I) will be taken up in an organic solvent to form a slurry, followed by the addition of aqueous acid. Preferred solvents for this purpose include polar solvents, such as acetates, including the acetates discussed above. n-Butyl acetate is particularly preferred.

The reaction may be continued for a suitable period of time, for example, from about 0.5 to about 5 hours, preferably for about 1 to 3 hours, more preferably about 1.5 hour. The reaction may be monitored by standard analytical techniques; such as thin layer HPLC. After the deprotection has proceeded to the desired level of completion, the compound of formula (VI) may be isolated from the reaction medium.

In preferred embodiments, this isolation of compound (V) may involve adjusting the pH of the aqueous mixture with a suitable base to a basic pH. If an organic solvent is used during the deprotection, it may be necessary to add an additional solvent. By way of illustration, if butyl acetate is used during deprotection, the addition of, for example, ethyl acetate may be appropriate. A wide variety of bases may be used for adjusting the pH of the aqueous solution including, for example, sodium hydroxide (NaOH), sodium bicarbonate (NaHCO₃) and sodium carbonate (Na₂CO₃).

After the pH has been adjusted to a basic pH, the compound of formula (V) may be removed from the aqueous medium, for example, by extraction with a suitable organic solvent. A wide variety of extraction solvents may be used, with halogenated solvents being preferred. Preferred among the halogenated solvents include methylene chloride (CH₂Cl₂) and chloroform (CHCl₃), with CH₂Cl₂ being more preferred. If, however, an organic solvent was employed the addition of solvent may be unnecessary as the product will likely reside in the organic layer following neutralization with base. In such a case, the layers may simply be separated and the organic layer washed with water to remove residual salts. The compound of formula (V) may then be isolated, for example, by removing the extraction solvent in vacuo.

Alternatively, the extraction solvent may be replaced with a second solvent, or combination of solvents, which is different from the extraction solvent and from which the compound of formula (V) may advantageously crystallize. For example, the extraction solvent may be substantially or completely removed in vacuo, followed by replacement of the extraction solvent with a crystallization solvent. As will be appreciated by one of ordinary skill in the art, a wide variety of crystallization solvents, including combinations thereof, may be used for this purpose.

It has been surprisingly discovered that compound (V) may possess beneficial properties, such as high bioavailability and minimal gastric effects, depending upon the particular particle form. For example, in certain embodiments, smaller particles, for example, those having an average particle diameter of, for example, about 40 μm or less, more preferably about 30 μm or, less, even more preferably about 20 μm or less, are preferred. Methods to obtain such smaller particle sizes may include, for example, milling or micronization. Alternatively, smaller particles may be produced directly by crystallization engineering, wherein parameters such as solvent, temperature, and seeding are controlled to produce a preferred form. Various solvents described herein, including combinations thereof, i.e., solvent systems containing two or more solvents, may be used to produce such preferred crystals.

It has also been discovered that certain solvents, such as acetates, present in the crystallization solution may prevent the formation of larger crystals. Thus, in certain embodiments, the use of acetates, such as ethyl acetate, alone or in combination with other solvents is preferred as a crystallization solvent. In other embodiments, acetonitrile is the preferred solvent. In still other embodiments, an alcohol solvent, alone or in combination with other solvents, may be preferred. When alcohol solvents are used, the product is preferably crystallized from ethanol, containing a small percentage of additional solvents. As will be appreciated by one of ordinary skill in the art, numerous solvents may be used in combination with ethanol, including, but not limited to other alcohols such as methanol, propanol, isopropanol, and t-butanol; ketones such as acetone and methyl isobutyl ketone (MIBK); aromatic solvents such as benzene, toluene, and xylene; and acetates such as methyl acetate, ethyl acetate, isopropyl acetate, and butyl acetate. In addition to physical quality, the choice of solvent may also involve other process considerations such as, for example, the desire to have a product devoid of residual toxic solvent. Thus, in certain embodiments, volatile solvents, i.e., those with boiling points below 100° C., are preferred. In a particularly preferred embodiment, an ethanol solvent system from which the product is crystallized contains isopropanol and acetate. In a more preferred embodiment, the ethanol solvent system contains about 1% to about 10% isopropanol and about 1% to about 50% ethyl acetate. In an even more preferred embodiment, the amount of isopropanol is about 2% to about 7% and the amount of ethyl acetate is about 1% to about 5%. In another preferred embodiment, the acetate is residual from the reaction work-up and the amount thereof is controlled by distillation.

The present invention may be further exemplified without limitation by reference to Scheme 4.

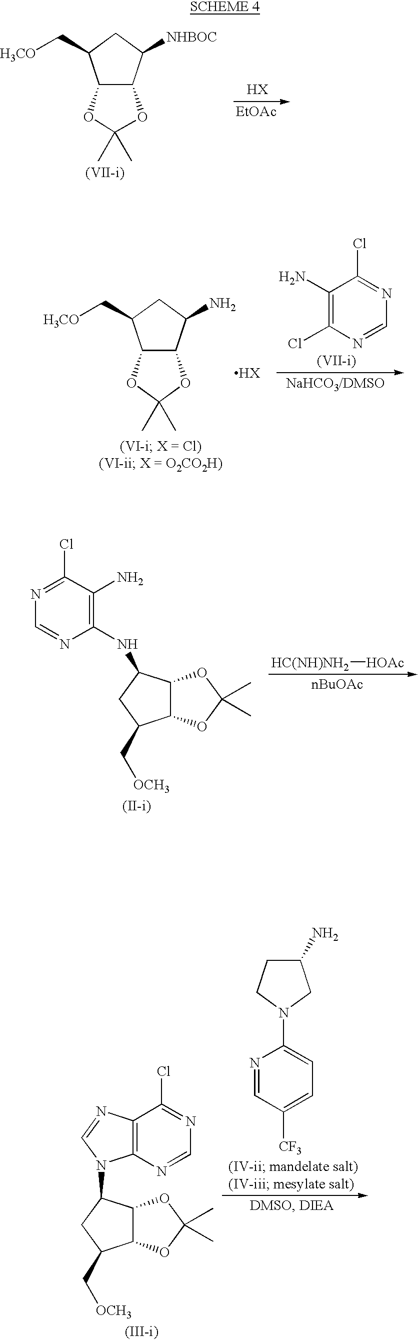

The invention is further described in the following examples. All of the examples are actual examples. These examples are for illustrative purposes only, and are not to be construed as limiting the appended claims.

### EXAMPLE 1

This example describes the preparation of the protected carbosugar (i) using methods within the scope of the present invention.

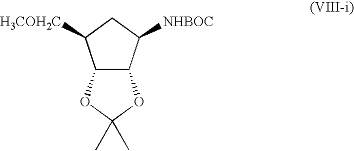

A. Derivatization of Lactam (ii) with Di-tert-butyldicarbonate

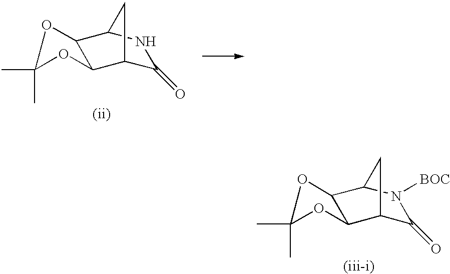

Solid lactam (ii) (200 g, 1.09 mole) and 4-dimethylaminopyridine (3.32 g, 0.027 mole) were charged to a 4 L reactor. Toluene (1000 mL) was added and the mixture was stirred at moderate speed while heating to 35° C. Di-tert-butyldicarbonate (TBOC, 260.8 g, 1.19 mole) was then added dropwise over 30 minutes. After completion of the reaction (ca. 2 h), the mixture was used as is in the next step (i.e., without further work-up or purification).

B. Reductive Lactam Opening of (iii-i)

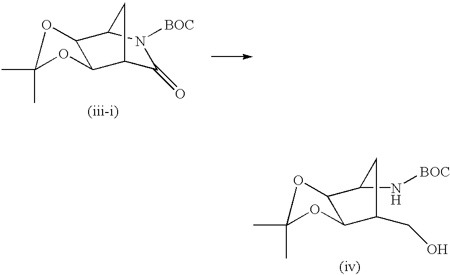

The process stream from Step A was charged to a 4 L reactor and the solution was cooled to 10-15° C. while being stirred at moderate speed. The reactor was charged with granular sodium borohydride (41.2 g, 1.09 mole) and methanol (34.88 g, 1.09 mole). After the exotherm subsided, the mixture was heated to 20-25° C. After completion of the reaction (ca. 24 h), the mixture was washed with 5% citric acid (200 mL) and water (3×150 mL). The aqueous layers were combined and back extracted with toluene (200 mL). The toluene layers were combined and used as is in the next step (i.e., without further work-up or purification).

B′. Alternate Lactam Opening of (iii-i)

In an alternate procedure, the process stream from Step A (874.4 g, 0.713 mol) was charged to a 1 L reactor and the solution was cooled to 10-15° C. while being stirred at moderate speed. The reactor was charged with a 2 M solution of lithium borohydride in THF (177.8 mL, 0.355 mole) over 1 hr. An exotherm from 13° C. to 21° C. was observed. Once the addition was complete, the mixture was heated to 20-25° C. After completion of the reaction (ca. 1 h), the mixture was quenched with water (263 mL) and stirred for 30 min. The agitated mixture was discharged from the reactor onto a filtration funnel. The filter cake was washed with toluene (2×200 mL), then the wash and the filtrate were charged back into the reactor. The phases were separated. The organic phase was cooled to 1.5° C., 1 N HCl (260 mL) was added over 15 min., then the mixture was stirred for 3 minutes. The layers were separated and the organics were diluted with water (260 mL) over 30 min. at 7° C. and the mixture was stirred for 5 minutes, then the phases were separated. The cold aqueous phases were combined and back-extracted with toluene (76.8 g). The back extraction toluene phase was added to the main toluene pool, which was kept at <10° C. for use “as is” in the next step (i.e., without further work-up or purification).

C. Methylation of (iv) with Dimethylsulfate

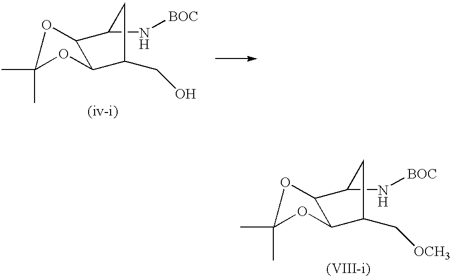

The process stream from Step B and 50% aqueous sodium hydroxide (400 g, 5.0 mole) were charged to a 4 L reactor. The solution was cooled to 10-15° C. while being stirred at moderate speed. The reactor was charged with dimethylsufate (200 g, 1.58 mole) dropwise over 30 minutes. After the exotherm subsided, the mixture was heated to 20-25° C. After completion of the reaction (ca. 2 h), the mixture was treated with ammonium hydroxide (200 mL) and was allowed to react for 30 minutes. The layers were split and the aqueous phase was discarded. The toluene layer was washed with water (250 mL). The layers were split and the aqueous phase was discarded. The toluene layer was diluted with propylene glycol (1000 mL) and the toluene was distilled under reduced pressure. After the distillation was completed, the glycol solution was treated with water (1500 mL) and the product was allowed to crystallize at 5° C. The solids were collected by filtration and washed with water (2×1000 mL). The solids were dried to constant weight in a vacuum oven at 50° C. to provide the methylated product (i) as a white solid (280 g, 0.93 mole, 85% for 3 steps).

### EXAMPLE 2

This example describes the preparation of 1-(5-trifluoromethyl-pyridin-2-yl)-pyrrolidin-3(S)-ylamine (IV) using methods within the scope of the present invention.

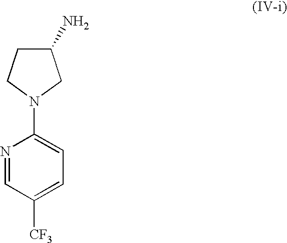

A. Condensation of 3(R)-hydroxypyrrolidine (vii) with 2-Chloro-5-trifluoro-methylpyridine (viii) to yield 1-(5-Trifluoromethyl-pyridin-2-yl)-pyrrolidin-3(R)-ol.

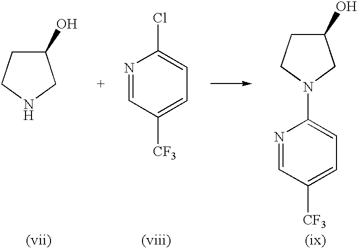

2-Chloro-5-trifluoromethylpyridine (viii) (90.9 g, 0.5 mol), ethanol (200 mL), 3(R)-hydroxypyrrolidine hydrochloride, (vii) (61.8 g, 0.5 mol), potassium carbonate (82.9 g, 0.6 mol) and water (50 mL) were charged to a reactor vessel. The mixture was heated at 78° C. for 24 hours, after which ion pair chromatography showed consumption of starting material. At 78° C., water (350 mL) was charged to the reaction, and the mixture was held at 78° C. for 1 hour. The reaction was cooled to 20° C., then the precipitate was isolated by filtration. The filter cake was washed with water (300 mL), then dried in a vacuum oven at 50° C. with nitrogen bleed overnight. The reaction yielded 1-(5-trifluoromethyl-pyridin-2-yl)-pyrrolidin-3(R)-ol (ix) as a white solid (93.5 g, 80.5% yield).

A′. Alternate Preparation of (ix)

In an alternate procedure, DMSO (3.1 Kg) was charged to a reaction vessel and heated to 40° C. 3R-Hydroxypyrrolidine hydrochloride (0.68 Kg, 5.5 moles) was then added to the vessel, followed by potassium carbonate (0.92 Kg, 6.7 moles) and melted 2-chloro-5-trifluoromethylpyridine (1.0 Kg, 5.5 moles). The reaction mixture was heated to 100° C. over 50 minutes and held at that temperature for 3 h. When the reaction was judged complete, it was cooled to 30-35° C., water (4.2 Kg) was added, and the mixture was cooled to 20° C. The resulting solid was filtered and washed with water (2×3.2 Kg) and dried to afford 1-(5-trifluoromethyl-pyridin-2-yl)-pyrrolidin-3(R)-ol (ix) (1.2 Kg, 95.9% stoich. yield).

B. Conversion of 1-(5-Trifluoromethyl-pyridin-2-yl)-pyrrolidin-3(R)-ol (ix) to Methanesulfonic acid 1-(5-trifluoromethyl-pyridin-2-yl)-pyrrolidin-3(R)-yl ester (x).

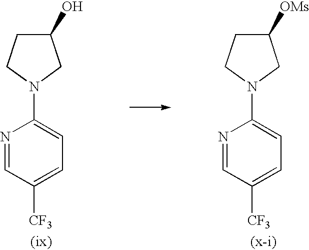

1-(5-Trifluoromethyl-pyridin-2-yl)-pyrrolidin-3(R)-ol (ix) (50 g, 216 mmol) and dichloromethane (500 mL) were charged to a reactor, and the solution was stirred at moderate speed. Triethylamine (39 mL, 280 mmol) was added to the reactor, and the solution was cooled to 5° C. Methanesulfonyl chloride (22 mL, 280 mmol) was then added to the reactor over a 20 minute period, and the resulting exotherm initially raised the temperature to 18° C. The rate of addition was controlled so as to keep the internal temperature below 18° C. After addition of methanesulfonyl chloride was complete, the mixture was stirred for 5 minutes during which time the internal temperature fell to 9° C. The mixture was allowed to warm to 23° C. over 2 h, and the formation of a solid precipitate (Et₃N.HCl) was observed. The reaction was judged complete by TLC and LC. The mixture was washed with sat. NaHCO₃ (300 mL), and the aqueous layer was back-extracted with CH₂Cl₂ (100 mL). The combined organic extracts were washed with brine (200 mL), and the organic extracts were re-charged to the reactor. Dichloromethane was distilled to the MSV (jacket 55° C., internal 43° C., head 39° C.; 425 mL collected, estimated 150 mL in reactor). The reactor was cooled to 35° C. and charged with 2-propanol (300 mL). The internal temperature cooled to 30° C., then raised back to 35° C. The solvent ratio in the reactor was 80/20 2-propanol/CH₂Cl₂. Solid started to precipitate in the reactor, and the mixture was stirred very fast and was cooled to ambient temperature overnight. The mixture was cooled to 0° C., then filtered. The filter cake was washed with 2-propanol (2×100 mL), then dried in a vacuum oven at 40° C. under 15” Hg with a nitrogen bleed for 18 h to afford methanesulfonic acid 1-(5-trifluoromethyl-pyridin-2-yl)-pyrrolidin-3(R)-yl ester (x-i) (54.95 g, 83%) as a white solid.

C. Conversion of Methanesulfonic acid 1-(5-Trifluoromethyl-pyridin-2-yl)-pyrrolidin-3(R)-yl ester (x) to Benzyl-[1-(5-trifluoromethyl-pyridin-2-yl)-pyrrolidin-3(S)-yl]-amine (xi).

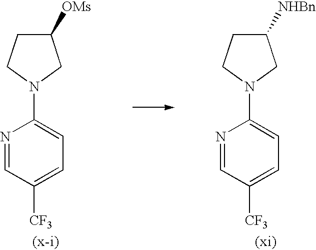

Benzylamine (122.6 g, 1.14 mol) and methanesulfonic acid 1-(5-trifluoromethyl-pyridin-2-yl)-pyrrolidin-3(R)-yl ester (50.1 g, 0.162 mol) were charged to a 5 L reaction vessel. The reaction mixture was warmed to 110° C. (a clear solution was formed between 40-50° C.). The mixture was held at 110° C. for 6.5 h, at which point HPLC showed 0.5% of starting material. The mixture was cooled to 20° C., then methyl-tert-butyl ether (MTBE; 300 mL) followed by saturated aqueous sodium bicarbonate (500 mL), were added and the mixture was stirred. The phases were separated and the organic layer was washed with brine (500 mL). The phases were split, then the organic layer was diluted with MTBE (600 mL), after which carbon dioxide (162.2 g, 3.69 mol) was bubbled into the solution for 3 hours at room temperature. A precipitate formed during the addition, and was removed by filtration. The filtrate was transferred to a distillation vessel; residues in the filtration flask were washed out with MTBE (2×50 mL) and added to the main MTBE pool. MTBE (˜325 mL collected) was minimized by distillation at atmospheric pressure over 2.5 hours. The remaining solution was cooled to 25° C., then diluted with ethanol (500 mL). The remainder of MTBE (˜225 mL collected) was fractionally distilled off at atmospheric pressure to achieve a ratio of 85/15 ethanol/MTBE. The solution was cooled to room temperature and was used as is in the next step (i.e., without further work-up or purification).

D. Hydrogenolysis of Benzyl-[1-(5-trifluoromethyl-pyridin-2-yl)-pyrrolidin-3(S)-yll-amine (xi) to 1-(5-Trifluoromethyl-pyridin-2-yl)-pyrrolidin-3(S)-ylamine (IV-i).

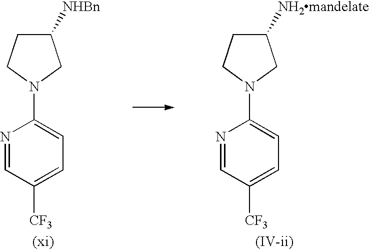

To a reaction vessel were added benzyl-[l-(5-trifluoromethyl-pyridin-2-yl)-pyrrolidin-3(S)-yl]-amine (xi) (51.9 g, 162 mmol), ethanol (350 mL), and methyl-tert-butyl ether (100 mL). Glacial acetic acid (46.5 mL) was added to the stirred, room temperature mixture over 1 minute, raising the temperature from 23° C. to 27° C. The solution was stirred for 10 minutes, then a slurry of 5% Pd/C 50% wet (10.3 g) in ethanol (50 mL) was added. The mixture was warmed to 65° C. +/−2° C. and ammonium formate (30.6 g, 0.485 mol) was added which cooled the internal temperature to 58° C. The mixture was rewarmed to 65° C. and held there with stirring for 1.5 h. Gas evolution occurred early in the 1.5 hour period, but subsided; ion pair chromatography showed 87% product. Glacial acetic acid (10.2 mL) and ammonium formate (10.2 g) were recharged to the reactor, the temperature was re-established at 65° C., and gas evolution resumed. One hour after the recharge, the reaction was 92% complete. The mixture was allowed to cool to room temperature overnight; HPLC showed the reaction to be 99% complete the following morning. The catalyst was removed by filtration through glass microfibre paper. The solids were washed with ethanol (200 mL) with care taken to keep the catalyst bed wet. The combined organics were distilled to a minimum volume in which stirring could be maintained, collecting about 600 mL ethanol. While the solution was still hot (85° C.), ethyl acetate (2×125 mL) was added and the mixture was distilled, azeotropically removing EtOH/EtOAc. While the mixture was still warm (85° C.), ethyl acetate (250 mL) followed by water (200 mL) were added. The mixture was cooled to ca. 25° C., (phase separation occurred), then the pH of the aqueous phase was adjusted to pH 11 with NaOH (40 g NaOH in 80 mL water). The mixture was stirred until the pH was stable at pH 11, then stirring was stopped and the phases were separated. The aqueous phase was extracted with ethyl acetate (2×200 mL).

The organic layers were combined and (+)-mandelic acid (24.6 g, 162 mmol) was added. The mandelic acid dissolved, and after approximately 10 minutes, a solid precipitated. The mixture was heated causing dissolution of the precipitate once the internal temperature reached 73° C. The solution was distilled until 300 mL of distillate was collected. After 200 mL was collected, a solid re-precipitated. Ethyl acetate (300 mL) was added to the mixture which was cooled to 3° C. and held there for 30 min. The solids were collected by filtration. The filter cake and reactor were washed with ethyl acetate (100 mL). The filter cake was dried in a vacuum oven at 40° C. overnight to yield 1-(5-trifluoromethyl-pyridin-2-yl)-pyrrolidin-3(S)-ylamine mandelate salt (41.6 g, 109 mmol, 67% yield) as a white solid.

D′. Alternate Preparation of (IV-ii) from 3S-acetamidopyrrolidine (xii-i) and 2-chloro-5-trifluoromethyl pyridine (viii).

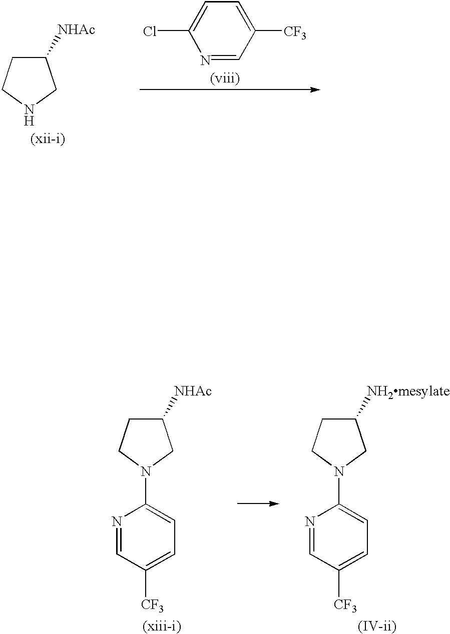

3S-Acetamidopyrrolidine (xii-i) (213.4 g, 1.65 mole) and 2-chloro-5-trifluoromethyl pyridine (xiii) (282.8 g, 1.55 mole) were melted in a hot water bath (80° C.) and poured into a 4 neck, 3 L round bottom flask. The amber mixture was diluted with ethanol (0.32 L), then triethylamine (0.66 mL, 4.71 mole) was added to the stirred mixture in a steady stream over 0.25 h. The stirred mixture was heated at reflux for 20 h. A crystalline material accumulated along the walls of the vessel, but agitation of the main mixture was unimpeded and the solid material was easily dislodged back into the stirred mixture. In process control analysis (HPLC, 50 μL reaction mixture in 1.5 mL MeOH, 10 μL injection) showed a ratio of N-[1-(5-trifluoromethyl-pyridin-2-yl)-pyrrolidin-3-yl]-acetamide to (xiii) of 16.3:1. The reaction mixture was heated near reflux for an additional overnight period (total ca. 48 h). The mixture was cooled to 5° C., then concentrated hydrochloric acid (1 L) was added slowly, resulting in the formation of “snowflakes” in the reactor. The brown mixture was heated at boiling as ethanol was distilled away. The pot temperature rose from 85° C. to ca. 105° C. as 500 mL of distillate were removed over a 1.5 h period. Distillate collection was stopped, and the mixture was heated at reflux for another 3.5 h.

In process control analysis (HPLC, 50 μL reaction mix in 1.5 mL methanol, 10 μL injection) showed a trace of starting material. Heating was removed and the mixture was stirred as it cooled to room temperature. The mixture was poured into water (1.2 L). With the vessel jacket at 10° C. and stirred pot contents at 22° C., 10 N sodium hydroxide (400 mL, 4 mole) was added slowly, causing a temperature rise from 22° C. to 44° C. Product began to precipitate; ethyl acetate (1 L) was added (pot went to 33° C.), then 10 N sodium hydroxide (0.44 L, 4.36 mole) was added slowly with little exothermic impact. After addition, the phases were split (1.62 L organic phase collected), and ethyl acetate (1 L) was added, followed by agitation (10 min.) and phase separation (5 minutes, clean; 1 L org phase collected). A final extraction with ethyl acetate (0.6 L) gave 0.6 L of recovered organic phase.

The combined organic extract was washed with brine (1 L); complete phase separation was slow, requiring overnight. The organic solution was heated (jacket at 110° C.) to distill triethylamine and water: in three cycles, 0.5 L distillate was collected and fresh ethyl acetate (0.5 L) was added to the pot. A final 650 mL of distillate was collected, the mixture was then cooled to 10° C., diluted with ethyl acetate (1 L), and treated dropwise with methanesulfonic acid (.153.8 g, 1.6 mole) over 45 minutes. White crystals formed immediately upon the initiation of the methanesulfonic acid addition, and the mixture became thick, but stirring was able to be maintained. The mixture was stirred at 10° C. for 1 h after all of the methanesulfonic had been added, then was filtered (Whatman #2 paper, 12 minutes). The filter cake was washed with ethyl acetate (1 L; 20 min. filtration time),. then the cake was air dried (576 g), then dried under vacuum with a N₂ bleed, at 45° C. overnight, yielding 422 g (1.29 moles, 82.7%) of 1-(5-trifluoromethyl-pyridin-2-yl)-pyrrolidin-3(S)-ylamine methanesulfonic acid salt. MS: M_{calc}, 231; M_{obsvd}, 232. NMR: 2.12 (m, 1H), 2.35 (m, 1H), 2.38 (s, 3 H), 3.5 (m, 1H), 3.6 (m, 2H), 3.73 (dd, 1H), 3.9 (m, 1H), 6.78 (dd, 1H), 7.82 (dd, 1 H), 8.4 (s, 1H).

D″. Alternate Preparation of (IV) from 3S-aminopyrrolidine and 2-chloro-5-trifluoromethylpyridine.

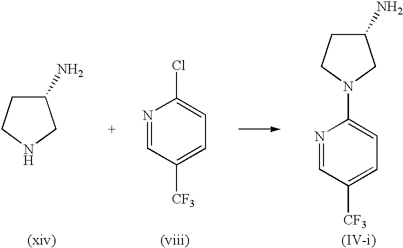

2-Chloro-5-trifluoromethylpyridine (20 g, 0.11 mol), 2-S-aminopyrrolidine (10.74 g, 0.124 moles), potassium carbonate (22.8 g, 0.151 moles) and ethanol (200 mL) were charged to a reaction vessel. The mixture was heated at 78° C. for 21 h, then filtered hot. The filtrate was cooled to room temperature, then (+)-mandelic acid (16.73 g, 0.109 moles) was added. A solid formed immediately. The solid was filtered and dried under vacuum at 50° C. A second crop was obtained from the filtrate. The first crop gave 23.49 g (56% stoich. yield) of 1-(5-trifluoromethyl-pyridin-2-yl)-pyrrolidin-3(S)-ylamine with an ee of 99% S . The second crop was 0.3 g, (1% stoich.yield).

The above procedure was repeated except that methane sulfonic acid (10.74 g, 7.07 mL, 0.113 moles) was used in place of (+)-mandelic acid. A first crop that formed was filtered, then dried under vacuum at 50° C. to afford 30.23 g (84% stoich. yield) of 1-(5-trifluoromethyl-pyridin-2-yl)-pyrrolidin-3(S)-ylamine with 97% ee. A second crop was isolated of 1.43 g (4% stoich. yield) was likewise isolated.

### EXAMPLE 3

This example describes the preparation of (1R,2S,3R,5R)-5-methoxymethyl-3-[6-[1-(5-trifluoromethyl-pyridin-2-yl)pyrrolidin-3(S)-ylamino]-purin-9-yllcyclopentane-1,2-diol] (V-i) using the materials obtain from Examples 1 and 2 and methods within the scope of the present invention.

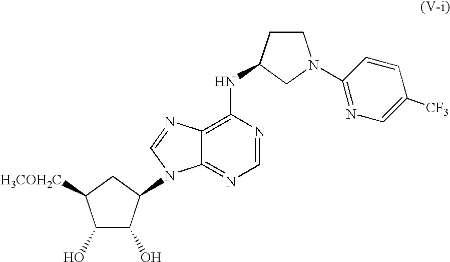

A. Deprotection of Protected Carbosugar (VII-i) to Afford 2(S),3(R)-isopropylidenedioxy-4(S)-methyoxymethylcyclopentan-1(R)-amine hydrochloride salt (VI-i)

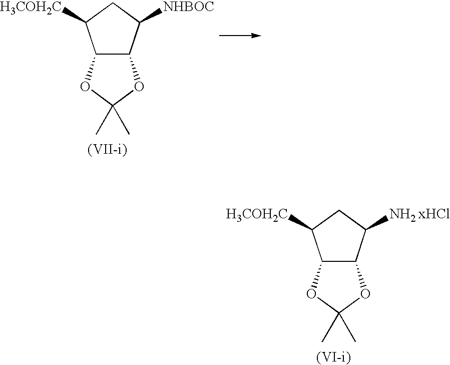

Hydrochloric acid (ca. 250 g, 6.8 mol, approximately saturated solution) was added via diptube over approx. 2 hours to a chilled (10° C.) suspension of the protected carbosugar (VII-i) (452.1 g, 1.5 mol) in anhydrous n-butyl acetate (1.9 L) and under nitrogen. The temperature was allowed to increase to 25° C. during this addition. After an additional 1 hour at rt, TLC analysis (1:1 heptane/ethyl acetate, ninhydrin spray) showed that the reaction was complete. The reaction solution was degassed using vacuum in combination with nitrogen purging for 1 h. The resulting n-butyl acetate solution of 2(S),3(R)-isopropylidenedioxy-4(S)-methyoxymethylcyclopentan-1(R)-amine hydrochloride (VI-i) was used as is in the next step (i.e., without further work-up or purification). For purposes of characterization, a small sample of the solution was concentrated under reduced pressure to give crude (VI-i) as a light yellow oil. Anal. Calcd. For C₁₀H₂₀ClNO₃: C, 50.52, H, 8.48, N, 5.89. Found: C, 49.33, H, 8.50, N, 5.93. MS (ion spray), m/z 202 (M⁺+1, 100%). ¹H-NMR (500 MHz, DMSO-d6, δ ppm) 8.6 (broad s, 3H), 4.58 (m,1H), 4.41 (m, 1H),3.4-3.2 (total 6H), 2.25 (m, 2H), 1.65 (m, 1H), 1.42 (s, 3H), 1.25 (s, 3H). Approximately 10% of the corresponding diol was present in the crude material.

A′. Alternate Deprotection Procedure

Step A was repeated except that EtOAc was substituted for n-BuOAc. Specifically, HCl (26 g, 0.7 mol, approximately saturated solution) was added via diptube over approx. 30 min to a chilled (10° C.) suspension of the protected carbosugar (28.6 g, 94.8 mmol) in anhydrous EtOAc (129 mL) and under nitrogen. The temperature was allowed to increase to 25° C. during this addition. After an additional 1 hour at rt, TLC analysis (1:1 heptane/ethyl acetate, ninhydrin spray) showed that the reaction was complete. The reaction solution was degassed using vacuum in combination with nitrogen purging for 1 h. This operation resulted in the reduction of the reaction mixture volume by approximately 65%. The resulting mixture was used as is in the next step (i.e., without further work-up or purification).

A″. Salt Formation

The free base of the compound of formula (VI) (8 g dissolved in acetonitrile to provide 80 mL stock solution) was treated with various acids to afford the corresponding salts . of 2(S),3(R)-isopropylidenedioxy-4(S)-methyoxymethylcyclo-pentan-1(R)-amine. Polarizing light microscopy showed the crystallinity of each of the isolated salts.

Eight grams of (VI) was dissolved in acetonitrile to provide 80 mL of a first stock solution.

D-tartrate (VI-iii): Ten mL of the first stock solution (containing 1.00 g (VI)) was added to a solution of 0.69 g D-tartaric acid in 60 mL acetone. Solids formed on standing overnight, which were isolated by filtration, giving 1.5 g of a white solid, m.p. 115-116° C. Analysis: calculated for C₁₄H₂₅NO₉, C, 47.86; H, 7.17; N, 3.99. Found C, 45.69; H, 7.31; N, 3.71.

Succinate (VI-iv): One mL of the first stock solution was added to a solution of 27 mg succinic acid in 5 mL acetone. After standing overnight, the crystals that formed were isolated by filtration and washed with additional acetone to yield 62 mg of a white flaky solid, m.p. 138-139° C. Analysis indicated that this protocol afforded two molecules of the salt of (VI) with one molecule of succinic acid. A second crop of 21 mg was isolated from the mother liquors. ¹H NMR (DMSO-d₆) δ 4.35 (dm), 3.3 (m), 3.25 (s, —OCH₃), 2.5 (s, —NH₃), 2.35 (s, succinic acid —CH₂—), 2.2 (m), 1.3 (m), 1.4 and 1.2 (singlets, acetonide —CH₃). Analysis: calculated for C₂₄H₄₄N₂O₁₀, C, 55.37; H, 8.52; N, 5.38. Found C, 55.43; H, 8.68; N, 5.35.

L-tartrate (VI-v): One mL of the first stock solution was added to a solution of 0.069 g L-tartaric acid in 10 mL acetone. The solids that formed on standing overnight were isolated by filtration and washed with tert-butyl methyl ether (TBME) to give 0.110 g of white crystals, m.p. 100-102° C. ¹H NMR (DMSO-d₆) δ 4.4 (m, 2H), 3.85 (s, 2H), 3.35 (m, 3H), 3.25 (s, 3H), 2.2 (m, 2H), 1.5 (m, 1H), 1.38 (s, 3H), 1.20 (s, 3H) (—NH₃ protons not shown). Analysis: calculated for C₁₄H₂₅NO₉, C, 47.86; H, 7.17; N, 3.99. Found C, 46.24; H, 7.29; N, 4.00.

Fumarate (VI-vi): One mL of the first stock solution was added to a solution of 54 mg fumaric acid in 10 mL acetone. The solvent was allowed to evaporate and the residue was triturated with TBME. The resulting solids were isolated by filtration to give 73 mg, m.p. 140-143° C. ¹H NMR (DMSO-d₆) δ 6.4 (s, 2H), 4.4 (m, 2H), 3.35 (m, 4H), 3.3 (s, 3H), 2.1 (m, 2H), 1.5 (m, 1H), 1.38 (s, 3H), 1.20 (s, 3H) (—NH₃ protons not shown). Analysis: calculated for C₁₄H₂₃NO₇, C, 52.99; H, 7.30; N, 4.40. Found C, 54.05; H, 7.64; N, 3.72.

One gram of (VI) was dissolved in TBME to give 20 mL of a second stock solution.

Formate (VI-vii): To 10 mL of the second stock solution was added 94 μL formic acid. The resulting solid was isolated by filtration to give 0.885 g of white solids, m.p. 115-117° C. ¹H NMR (DMSO-d₆) δ 8.3 (s, 1H, formate proton), 4.3 (dm, 2H), 3.4 (m, 3H), 3.2 (s, 3H), 2.1 (m, 2H), 1.4 (m, 1H), 1.3 (s, 3H), 1.15 (s, 3H) (—NH₃ protons not shown). Analysis: calculated for C₁₁H₂₁NO₅, C, 53.43; H, 8.56; N, 5.66. Found C, 53.41; H, 8.73; N, 5.62.

Benzoate (VI-viii): To 10 mL of the second stock solution was added a solution of 0.305 g benzoic acid in 5 mL TBME. On evaporation of the solvent, crystals formed. The resulting solid was triturated with fresh TMBE and isolated by filtration to give 0.55 g of white solids, m.p. 102-104° C. ¹H NMR (DMSO-d₆) δ 7.9 (m, 2H), 7.5 (m, 1H), 7.4 (m, 2H), 4.3 (dm, 2H), 3.4 (m, 1H), 3.25 (m, 2H), 3.2 (s, 3H), 2.1 (m, 2H), 1.35 (s, 3H), 1.3 (m, 1H), 1.15 (s, 3H) (—NH₃ protons not shown). Analysis: calculated for C₁₇H₂₅NO₅, C, 63.14; H, 7.79; N, 4.33. Found C, 63.13; H, 7.52; N, 4.11.

Dibenzoyl-D-tartrate (VI-ix): To 1 mL of the first stock solution was added a solution of 166 mg dibenzoyl-D-tartaric acid in 5 mL TBME. After standing overnight, the solids that formed were isolated by filtration to give 190 mg, m.p. 155-156° C. ¹H NMR (DMSO-d₆) δ 7.5 (m, 4H), 7.0 (m, 2H), 6.85 (m, 4H), 5.3 (s, 2H), 3.9 (dm, 2H), 2.9 (m, 3H), 2.8 (s, 3H), 1.9 (m, 2H), 1.05 (m, 1H), 0.85 (s, 3H), 0.65 (s, 3H) (—NH₃ protons not shown). Analysis: calculated for C₂₈H₃₃NO₁₁, C, 60.1; H, 5.94; N, 2.50. Found C, 57.50; H, 6.11; N, 2.28.

Dibenzoyl-L-tartrate (VI-x): To 1 mL of the first stock solution was added a solution of 166 mg dibenzoyl-L-tartaric acid in 5 mL TBME. After standing l0 overnight, the solids that formed were isolated by filtration to give 238 mg, m.p. 164-166° C. ¹H NMR (DMSO-d₆) δ 7.45 (m, 4H), 6.9 (m, 2H), 6.8 (m, 4H), 5.2 (s, 2H), 3.85 (dm, 2H), 2.8 (m, 3H), 2.7 (s, 3H), 1.8 (m, 2H), 1.0 (m, 1H), 0.75 (s, 3H), 0.6 (s, 3H) (—NH₃ protons, not shown). This material, shown by microscope to be amorphous, was crystallized from acetonitrile to give purer crystals. Analysis: calculated for C₂₈H₃₃NO₁₁, C, 60.1; H, 5.94; N, 2.50. Found C, 57.50; H, 6.11; N, 2.28.

Di-p-toluoyl-D-tartrate (VI-xi): To 1 mL of the first stock solution was added a solution of 179 mg di-p-toluoyl-D-tartaric acid in 5 mL TBME. After standing overnight, the solids that formed were isolated by filtration to give 238 mg, m.p. 183-185° C. ¹H NMR (DMSO-d₆) δ 7.15 (d, 4H), 6.45 (d, 4H), 5.1 (s, 2H), 3.7 (dm, 2H), 2.7 (m, 3H), 2.6 (s, 3H), 1.65 (m, 2H), 1.55 (s, 6H), 0.85 (m, 1H), 0.65 (s, 3H), 0.45 (s, 3H) (—NH₃ protons not shown). Analysis: calculated for C₃₀H₃₇NO₁₁, C, 61.32; H, 6.35; N, 2.38. Found C, 61.21; H, 6.27; N, 2.13.

Di-p-toluoyl-L-tartrate (VI-xii): To 1 mL of the first stock solution was added a solution of 179 mg di-p-toluoyl-L-tartaric acid in 5 mL TBME. After standing overnight, the solids that formed were isolated by filtration to give 215 mg, m.p. 193-195° C. ¹H NMR (DMSO-d₆) δ 7.20 (d, 4H), 6.50 (d, 4H), 5.1 (s, 2H), 3.75 (dm, 2H), 2.7 (m, 3H), 2.6 (s, 3H), 1.7 (m, 2H), 1.6 (s, 6H), 0.85 (m, 1H), 0.65 (s, 3H), 0.45 (s, 3H) (—NH₃ protons not shown). Analysis: calculated for C₃₀H₃₇NO₁₁, C, 61.32; H, 6.35; N, 2.38. Found C, 61.06; H, 6.41; N, 2.25.

In a similar manner, the first stock solution was also treated with malonic acid (VI-xiii), (−)-mandelic acid (VI-xiv) (as acetone solution), maleic acid (VI-xv) (as acetone solution), acetic acid (VI-xvi), glycolic acid (VI-xvii), trifluoroacetic acid (VI-xviii), salicylic acid (VI-xix) (as acetone solution), D-gluconic acid (VI-xx), methanesulfonic acid (VI-xxi), and p-toluenesulfonic acid (VI-xxii) (as acetone solution), phosphoric acid (VI-xxiii), and 30% hydrobromic acid in acetic acid (VI-xxiv) to form the corresponding salts of (VI).

B. Coupling of 2(S),3(R)-isopropylidenedioxy-4(S)-methyoxy-methylcyclopentan-i(R)-amine (VI-i) with 5-amino-4,6-dichloropyrimidine (VII) to Afford Coupled Product (II-i)

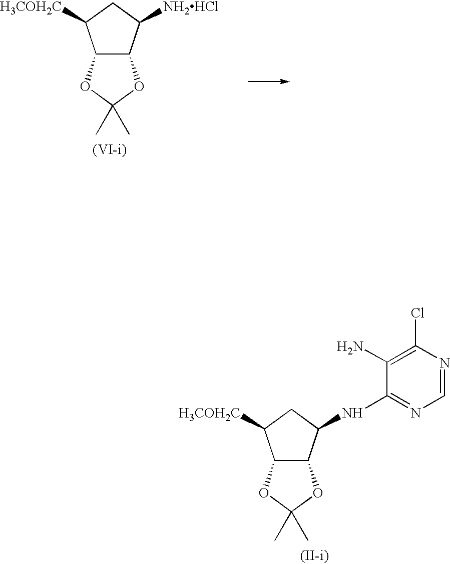

To the crude reaction mixture from Step A was added dropwise at room temperature diisopropylethylamine (679 g, 5.25 mol, 3.5 equiv.) then, in one portion, 5-amino-4,6-dichloropyrimidine (270.6 g, 1.65 mol, 1.1 equiv.). The reaction mixture was heated at 125° C. for 40 hours, after which analysis by HPLC (210 nm) showed 79% of the coupled product (II-i) and 13% 5-amino-4,6-dichloropyrimidine. After cooling to room temperature, the reaction mixture was washed successively with water (1.0 L) and 5% aqueous citric acid (1.0 L). The combined aqueous phases were back-extracted with 1.8 L of n-butyl acetate. Combined organics were washed with brine (0.5 L), then dried by azeotropic distillation of ca 200 mL of solvent. KF analysis of the solution showed 0.15% water. The resulting n-butyl acetate solution of the product was used as is in the next step (i.e., without further work-up or purification). For purposes of characterization, a small sample of this solution was concentrated under reduced pressure to give crude (II-i) as an oil. MS (ion spray), m/z 329, 331 (M⁺+1, Cl pattern, 100%). ¹H-NMR (500 MHz, CDCl₃, δ ppm) 8.1 (s, 1H), 6.5 (d, 1H), 4.65 (t, 1H), 4.58 (AB system, A portion, 1H), 4.38 (AB system, B portion, 1H), 3.62 (A1B1X system, A1 portion, 1H), 3.51 (A1B1X system, B1 portion, 1H), 3.47 (s, 3H), 3.33 (broad s, 2H), 2.70 (m, 1H), 2.32 (m, 1H), 1.56 (d, 1H), 1.42 (s, 3H), 1.25 (s, 3H)., The purity of the isolated oil, as assayed by HPLC (210 nm) was 83%.

B′. Alternate Coupling Procedure

Step B was repeated except that DMSO was substituted for n-BuOAc, and sodium bicarbonate was substituted for diisopropylethylamine, resulting in a significantly accelerated reaction rate. Specifically, to the crude reaction mixture from Step A were added DMSO (84 mL), followed by sodium bicarbonate (23.9 g, 284 mmol, 3.0 equiv.) and 5-amino-4,6-dichloropyrimidine (15.5 g, 94.8 mmol). The mixture was warmed to 105° C., while, distilling off some remaining EtOAc (reduced pressure was briefly applied to accelerate EtOAc removal). After stirring for 5 h at 105° C., the GC analysis showed 86% (by area) of the product (II-i) and less than 2% of the starting material (VI-i). The, mixture was cooled to room temperature, then partitioned between n-butyl acetate (320 mL) and water (940 mL). Phases were separated and the aqueous phase was reextracted with n-butyl acetate (130 mL). The combined organics were washed with brine (120 mL), then azeotropically distilled to remove water. The crude mixture was approximately 92% pure as assayed by HPLC at 210 nm, and was then used as is (i.e., without further work-up or purification) in the remainder of the synthesis (as described below, to give (V-i) with an overall yield of 52%).

B″. Alternate Coupling Procedure.

Step B′ was repeated except that the compound of formula (VI) was the oxalate salt (VI-ii) and was taken up in DMSO prior to addition to 5-amino-4,6-dichloro-pyrimidine via a syringe pump. Specifically, (VI-ii) (75.0 g, 257 mmol) was added to DMSO (118 g) and was stirred at ambient temperature until all solids dissolved. In a separate reactor, 5-amino-4,6-dichloropyrimidine (VII) (41.0 g, 249 mmol), sodium bicarbonate (76.25 g, 872 mmol), and DMSO were mixed together and heated to 80° C. The solution of (VII) was added via syringe pump over 70 minutes. Once the addition was complete, the temperature was raised to 105° C. over 15 minutes time. Heating was continued until HPLC analysis indicated that the level (A %, 265 nm) of (VII) dropped to <2% (typically 4 to 6 hours). The mixture was then cooled to 20° C. and butyl acetate (840 g) and water (2145 g) were added. The resulting solution was allowed to stir for 5 minutes and the layers were separated. The aqueous layer was back extracted with butyl acetate (327 g). The organic solutions were combined and were washed with 5% sodium chloride (416 g) solution. The butyl acetate solution was then concentrated by atmospheric distillation to achieve a solution of (II-i) which as approximately (9% w/w). Based upon assay, the yield for this example was 82%. This solution was used without purification in step C′.

C. Ring Closure of Coupled Product (II-i)

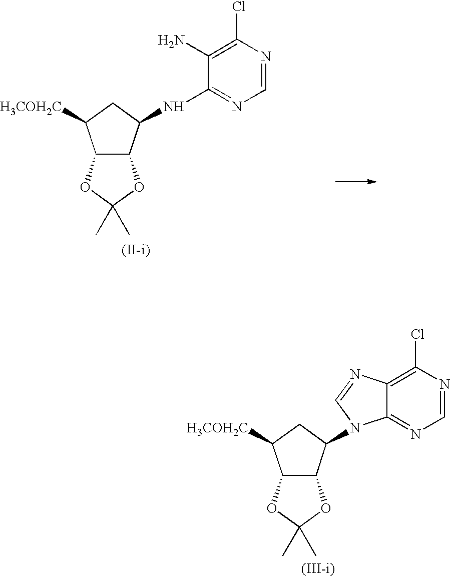

Formamidine acetate (624.7 g, 6.0 mol) was added to the reaction mixture from Step B. The mixture was heated at 124° C. for 3 hours, after which the HPLC analysis showed less than 2% (by area) of starting material (II-i) remaining. The reaction mixture was cooled to room temperature, then it was washed with water (1.2 L), 5% aqueous citric acid (1.0 L), and brine (1.0 L). The organic phase was azeotropically distilled under reduced pressure to remove water, then distilled until ca. 35% of the original volume was removed. The resulting n-butyl acetate solution showed 80% purity of the product (III-i) (HPLC, 210 nm, main impurity: 5-amino-4,6-dichloropyrimidine carried over from previous step) and was used as is in the next step (i.e., without further work-up or purification). For purposes of characterization, a small sample of the crude mixture was purified by flash chromatography (heptane/EtOAc, 1:1) to give (III-i) as a yellow oil. Anal. Calcd. For C₁₅H₁₉ClN₄O₃: C, 53.18, H, 5.65, N, 16.54. Found: C, 52.96, H, 5.68, N, 16.36. MS (EI), m/z 339, 341 (M⁺+1, Cl pattern, 100%). ¹H-NMR (500 MHz, CDCl₃, δ ppm) 8.73 (s, 1H), 8.22 (s, 1H), 5.02 (t, 1H), 4.88 (m, 1H), 4.69 (m, 1H), 3.55 (m, 2H), 3.40 (s, 3H), 2.55 (m, 2H), 2.42 (dd, 1H), 1.58 (s, 3H), 1.32 (s, 3H).

C′. Alternate Ring Closure of Coupled Product (II-i)

Step C was repeated except that the solution to which formamidine acetate was added was a DMSO solution obtained from alternate coupling procedure B″ above. Specifically, formamidine acetate (34.9 g) was added to a butyl acetate solution of (II-i) (308 g) prepared in Example B″. The mixture was then heated to 125° C. until HPLC analysis showed less than 2%, of starting material (II-i) (about 2 to 3 hours). The solution was allowed to cool down to room temperature and water was added. The mixture was stirred for 30 minutes and the layers separated. The organic layer was washed with 5% citric acid (58 g) and then with brine (59 g). The solution was concentrated under vacuum (200 mbar, pot temp. 77° C.) until about 150 g or butyl acetate had been collected. Dimethylsulfoxide (95 g) was added to the pot and distillation was continued until no more butyl acetate distilled off. The DMSO solution containing product (III-i) was used without further purification in step D′.

D. Coupling of (III-i) from Step C with 1-(5-trifluoromethyl-pyridin-2-yl)-pyrrolidin-3(S)-ylamine to Afford (I-i)

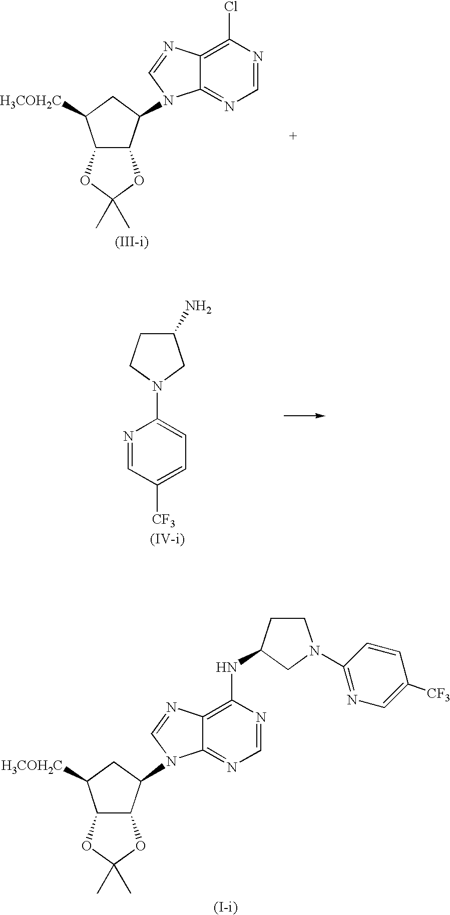

The methanesulfonic acid salt of 1-(5-trifluoromethyl-pyridin-2-yl)-pyrrolidin-3(S)-ylamine (IV-i) (425.5 g, 1.3 mol), followed by triethylamine (395 g, 3.9 mol) and ethanol (0.6 L) were added to the reaction mixture from Step C. The suspension was heated at 85° C. for 24 hours, after which HPLC analysis (210 nm, by area) showed 79.2% of the product (I-i) and 2.5% of the starting material (III-i). The reaction mixture was washed with water (1×2.5 L and 1×1.25 L), then used as is in the next step (i.e., without further work-up or purification). The purity of this crude mixture was assayed by HPLC (210 nm, by area) as 78%. For purposes of characterization, the crude mixture was purified by flash chromatography (CH₂Cl₂/MeOH, 95:5), to afford the product (I-i) as an off-white semi-solid. (The product can also be crystallized by adding its hot (ca. 85° C.) heptane solution into an approximately equal volume of cold (ca. 5° C.) heptane.) Anal. Calcd. For C₂₅H₃₀F₃N₇O₃: C, 56.28, H, 5.67, N, 18.38. Found: C, 55.22, H, 5.55, N, 17.72. MS (ion spray), m/z 534 (M⁺+1, 100%). ¹H-NMR (500 MHz, CDCl₃, δ ppm) 8.40 (s, 1H), 8.38 (m, 1H), 7.82 (s, 1H), 7.60 (m, 1H), 6.38 (d, 1H), 5.97 (broad d, 1H), 5.1-5.0 (m, 2H), 4.78 (m, 5 lines, 1H), 4.66 (dd, 1H), 3.96 (dd, 1H), 3.75-3.55 (3 overlapping multiplets, 3H), 3.53 (d, 2H), 3.40 (s, 3H), 2.52-2.35 (overlapping multiplets, 4H), 2.19 (m, 6 lines, 1H), 1.57 (S, 3H), 1.32 (S, 3H).

D′. Alternate Coupling of (III-i) from Step C′ with 1-(5-trifluoromethyl-pyridin-2-yl)-pyrrolidin-3(S)-ylamine to Afford (I-ii) (the mesylate salt of I-i)

Step D was repeated except that diisopropylamine was the base, DMSO was the solvent, and the compound of formula (III-i) was that prepared in Step C′ above. Specifically, (IV) and diisopropylethylamine (96.16 g) were added to a solution of (III-i) in DMSO (281 g) prepared in step C′. The reaction mixture was stirred and heated to 100° C. until HPLC analysis showed the level of (III-i) has fallen to <2 area % (265 nm) (about. 4 and 6 hours). The mixture was then cooled to room temperature and partitioned between butyl acetate (370 g) and water (1866 g). The organic phase was washed with 5% citric acid (3×178 g), 5% sodium bicarbonate solution (297 g), and brine (178 g). The resulting butyl acetate solution is assayed for content of (I) and was then azeotropically dried and concentrated to about 20-23% (I). This solution was then heated to 90° C., while a solution of methanesulfonic acid (4M in butyl acetate, 1.05 eq. 25.05 g) was quickly added, keeping the temperature >80° C. The solution was then cooled to 70° C. and was seeded with about 0.1 wt % (I-ii). The mixture was then cooled to about 10° C. and is stirred for 2 hours. The product was filtered and washed with butyl acetate. The salt was first dried under a nitrogen flow at room temperature and finally dried overnight in a vacuum oven at 80° C. (N₂ bleed, 20 in Hg) to provide (I-ii). Typical yields range from 78-95% yield. Anal. Calcd. for C₂₆H₃₄F₃N₇O₆S; C, 49.60; H, 5.44; N, 15.57; F, 9.05; S, 5.09. Found: C, 49.48; H, 5.57; N, 15.43; F, 8.92; S, 5.06. ¹H-NMR (300 MHz, MeOH-d₄, δ ppm) 8.40 (s, 2H), 8.3 (s, 1H), 7.9 (d, 1H), 6.9 (d, 1H), 5.1-4.8 (m, 5H), 4.6 (m, 1H), 4.1 (m, 1H), 3.8-3.6 (m, 3H), 3.5 (m, 2H), 3.4 (s, 3H), 3.3 (s, 1H), 2.65 (s, 3H), 2.6-2.2 (m, 5H), 1.5 (s, 3H), 1.25 (s, 3H).

D″. Salt Formation of (I-ii) From Compound (V)

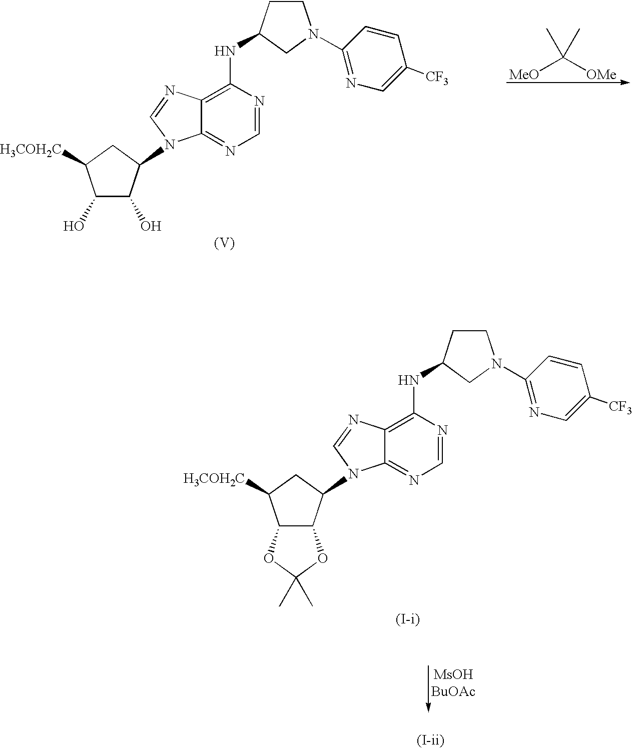

The compound of formula (I-ii) was prepared by treatment of crystalline (V) (9.9 g) with 2,2-dimethoxypropane (31.2 g) in acetone (200 mL), in the presence of p-toluenesulfonic acid monohydrate (7.6 g). After stirring overnight, the reaction mixture was neutralized with triethylamine (5.6 mL) and concentrated to dryness. The residue was dissolved in ethyl acetate (160 mL) and washed twice with aqueous sodium chloride. The resulting solution was concentrated to dryness to provide 11.1 g of (III-i) as a foamy amorphous solid. ¹H NMR analysis of (I-ii) (CDCl₃): d 8.40 (m, 2H), 7.82 (s, 1H), 7.60 (m, 1H), 6.38 (d, 1H), 5.98 (bd, 1H), 5.06 (b, 1H) overlapping with 5.04 (m, 1H), 4.77 (m, 1H), 4.66 (dd, 1H), 3.97 (dd, 1H), 3.55-3.75 (3 m, 3H), 3.52 (d, 2H), 3.40 (s, 3H), 2.7-2.52 (m, 4H), 2.18 (m, 1H), 1.58 (s, 3H), 1.32 (s, 3H).

D′″. Salt Formation of (III-i) From (I-i).

The free base of the compound of formula (I) (1.07 g, 2 mmol) was dissolved in butyl acetate (4.5 mL), and treated with one equivalent of methanesulfonic acid (0.13 mL) dissolved in butyl acetate (0.5 mL). The resulting salt began to precipitate almost immediately. The solids were dissolved upon heating. After cooling to room temperature, crystalline (III-i) was isolated by filtration. Drying under vacuum provided 0.845 g (67%) of RPR210966A as an off-white powder. ¹H NMR analysis of (III-i) (DMSO-d₆, 30° C.): d 9.4 (bs, 1H), 8.83 (bm, 1H), 8.48 (s, 1H), 8.41 (s, 1H), 7.85 (bd, 1H), 6.75 (b, 1H), 4.99 (quasi-t, 1H). 4.8-4.94 (bm, 2H), 4.55 (dd, 1H), 3.92 (bs, 1H), 3.70 (bm, 1H), 3.64 (bs, 1H), 3.44 (m, 2H), 3.29 (s, 3H), 2.42 (s, 3H), 2.34-2.4 (m, 2H), 2.2-2.3 (m, 2H), 1.49 (s, 3H), 1.23 (s, 3H); ¹³C NMR (DMSO-d₆, 80° C.): d 157.5 (C), 144.2 (CH), 134.2 (CH), 124.9 (q, CF₃), 118.9 (C), 112.73 (q, C—CF₃), 112.7 (C), 107.2 (CH), 83.3 (CH), 81.3 (CH), 73.2 (CH₂), 60.9 (CH), 58.2 (CH₃), 52.2 (C), 45.2 (CH₂), 43.2 (CH), 33.8 (CH₂), 30.6 (CH₂), 27.4 (CH₃), 25.2 (CH₃) (not all signals assigned).

D″″. Salt Formation of (III-i) From Isolated (I) In Solution

The concatenated reaction sequence A through C was used to prepare a solution containing approximately 20 wt% (I) in butyl acetate. To 4.5 mL of this liquor was added methanesulfonic acid (0.13 mL) dissolved in butyl acetate (0.5 mL). The resulting mixture was heated to dissolve precipitated salt, and seed crystals were added as the solution cooled. The crystalline (III-i) was isolated by filtration. Drying under vacuum provided 0.973 g (77%) of (III-i).

E. Deprotection of (I-i) from Step D

Aqueous HCl (3.0 N; 1.5 L) was added to the reaction mixture from Step D, and the resulting bi-phasic mixture was stirred for 1.5 hours at rt, after which the HPLC analysis (mixture of both phases) showed complete consumption of starting material. The phases were separated and the organic phase was discarded. Dichloromethane (3.2 L) was added to the aqueous phase, and the mixture was neutralized (pH 6-8) with 10 N aqueous NaOH (ca 0.5 L). The phases were separated, and the organic layer was washed with water (1.5 L), then filtered to eliminate a small amount of fine particles. Acetonitrile (4.8 L) was charged to the solution, and the bulk of dichloromethane was removed by distillation. Approximately 60% of the initial volume was removed, and the mixture temperature was approximately 75° C. at the end of the distillation. The solution volume was readjusted to ca 50% of the initial volume by adding acetonitrile (ca. 0.8 L), the mixture was warmed to reflux, then cooled to 20° C. over 4 hours. The crystalline product was isolated by filtration.

The filter cake was washed with acetonitrile (1.0 L then 0.5 L), then dried in vacuum oven with nitrogen bleed at 50° C. overnight to give (1R,2S,3R,5R)-3-methoxymethyl-5-[6-[1-(5-trifluoromethylpyridin-2-yl)pyrrolidin-3(S)-ylamino]-purin-9-yl]cyclopentane-1,2-diol] (I-i) (401.1 g, 54.2% overall yield) as a light beige solid of 99.7 (area) % purity as assayed by HPLC (265nm). M.p. 170° C. Anal. Calcd. For C₂₂H₂₆F₃N₇O₃: C, 53.55, F,11.55, H, 5.31, N, 19.87. Found: C, 53.60, F, 11.51, H, 5.16, N, 19.77. MS (ion spray), m/z 494 (M⁺+1, 100%). 1H-NMR (500 MHz, DMSO-d6, δ ppm) 8.38 (m, 1H), 8.26 (s, 1H), 8.20 (s, 1H), 7.87 (broad d, J=6.5 Hz, 1H), 7.71 (dd, J=9.0 Hz, 3.0 Hz, 1H), 6.56 (d, J=9.0 Hz, 1H), 5.01 (broad s, 1H), 4.86 (d, J=6.5 Hz, 1H), 4.74 (dt, J=10.0 Hz, 8.3 Hz, 1H), 4.63 (d, 4.5 Hz, 1H), 4.39 (m, 6 lines, 1H), 3.87 (m, 2H), 3.72 (m, 1H), 3.55 (m, 2H), 3.48 (ABX system, A portion, J=9.3 Hz, 6.3 Hz, 1H), 3.39 (ABX system, B portion, J=9.3 Hz, 6.3 Hz, 1H), 3.30 (s, 3H), 2.40-2.25 (2 overlapping multiplets, 2H), 2.21 (m, 2H), 1.78 (m, 8 lines, 1H).

E′. Alternate deprotection of (I-ii) from Step D′.

Step E was repeated except that the compound deprotected was formula (I-ii) prepared in Step D′ above. Specifically, (I-ii) (27.78 g) as a slurry in butyl acetate (67 g) was added, 3N HCl (91.7 g) and the mixture was stirred for 2 hours. The two phases were separated and ethyl acetate (250 g) was added to the acidic aqueous phase which was then neutralized with 10N NaOH (41.7 g) to pH 7.5±0.5. The aqueous layer was separated and the organic phase was was washed with deionized water (92 g). An azeotropic distillation reduced the ethyl acetate solution to about 17.5 wt % (V). Ethanol (49.6 g) was then added to afford 12.5 wt % solution and distillation was continued while maintaining a constant weight by addition of ethanol (235.5 g) until the concentration of ethyl acetate in the mixture was about 2%. The solution was cooled to 60° C. and seed crystals were added (0.5%). The batch was then cooled over 2-3 hours to room temperature. The resulting slurry was filtered using a Buchner funnel, rinsing the reactor and the filter cake with additional ethanol. The filter cake was dried under vacuum at 45° C. to give (V) as off-white crystals (17.48 g, 80.3% yield).

E″. Recrystallization of Compound (V)

To a 500-mL jacketed vessel were charged 30 g of (V) and 420 g of anhydrous ethanol. The mixture was heated to approximately 78° C., at which point all the solids dissolved. The resulting solution was cooled to 70° C. and a mixture of 50 mg micronized (V) in 0.5 mL ethanol was added as seeds. The solution was cooled gradually to 20° C. A portion of the resulting mixture was withdrawn, filtered using vacuum filtration, and dried under vacuum. The resulting solids were analyzed for particle size using a SYMPATEC device: x50 (50^{th} percentile size) of 137 μm, with a peak in the histogram at approximately 180 μm.

To the remaining suspension in the reactor was added 10 g ethyl acetate (to give a mixture containing approximately 4% ethyl acetate). The mixture was again heated until the solids had dissolved. The resulting solution was cooled to 60° C., at which point a slurry of micronized seeds was added as above. The mixture was cooled gradually to 20° C. A portion of the resulting mixture was withdrawn, filtered using vacuum filtration, and dried under vacuum. The resulting solids were analyzed for particle size using a SYMPATEC device: x50 (50^{th} percentile size) of 19 μm, with a single broad peak in the histogram centered at approximately 23 μm.

The disclosures of each patent, patent application and publication cited or described in this document are hereby incorporated herein by reference, in their entirety. Various modification of the invention, in addition to those described herein, will be apparent to those skilled in the art from the foregoing description. Such modifications are also intended to fall within the scope of the appended claims.

What is claimed is:

1. A process for the preparation of a compound of formula (I): 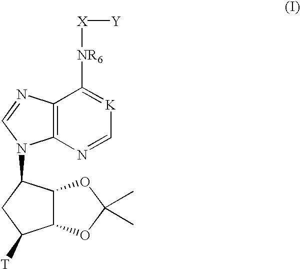
wherein:
K is N, N→O or CH;
R₆ is hydrogen, alkyl, allyl, 2-methylallyl, 2-butenyl or cycloalkyl;
X is 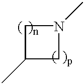
where the nitrogen of the ring of X is substituted by Y;
Y is hydrogen, alkyl, optionally substituted aralkyl, optionally substituted aryl, optionally substituted heterocyclyl or optionally substituted heterocyclylalkyl;
n and p are independently 0, 1, 2 or 3, provided that the sum of n and p is at least 1;
T is hydrogen, alkyl, acyl, thioacyl, halo, carboxyl, N(R₁)(R₂)C(═O), N(R₁)(R₂)C(═S) or R₃O—CH₂;
R₁, R₂ and R₃ are independently hydrogen, alkyl or cycloalkyl;
comprising:
contacting a compound of formula (II) 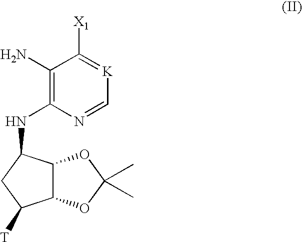
where X₁ is halo; with a formic acid derivative to provide a compound of formula (III): 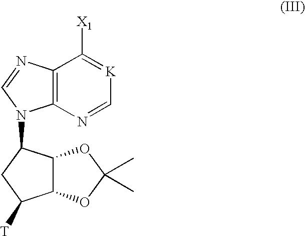
and contacting the compound of formula (III) with a compound of formula (IV)
R₆NH—X—Y (IV)
to provide the compound of formula (I) or a pharmaceutically acceptable salt form thereof.

2. A process according to claim 1 wherein:
K is N;
R₆ is hydrogen;
T is hydroxymethyl or methoxymethyl;
X₁ is chloro;
Y is optionally substituted heterocyclyl; and
the sum of n and p is 3 or 4.

3. A process according to claim 2 wherein:
T is methoxymethyl;
X is 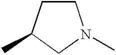
Y is optionally substituted pyridyl.

4. A process according to claim 3 wherein Y is 5-trifluoromethylpyrid-2-yl.

5. A process according to claim 4 wherein said formic acid derivative is selected from the group consisting of formamidine acetate, an orthoformate ester and dimethylformamide dimethyl acetal.

6. A process according to claim 5 wherein said formic acid derivative is formamidine acetate.

7. A process according to any of claims 1 to 6 further comprising deprotecting the compound of formula (I) to provide the compound of formula (V): 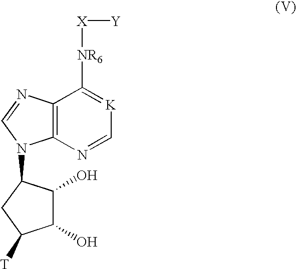
or a pharmaceutically acceptable salt form thereof.

8. A process according to claim 7 wherein said deprotection comprises contacting the compound of formula (I) with water containing at least two equivalents of an acid.

9. A process according to claim 8 wherein said acid is selected from the group consisting of HCl, HBr, H₂SO₄, HNO₃ and acetic acid.

10. A process according to any of claims 1 to 6 wherein the compound of formula (II) is prepared by a process comprising:
contacting a compound of formula (VI) 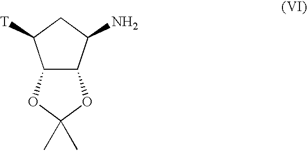
with a compound of formula (VII) 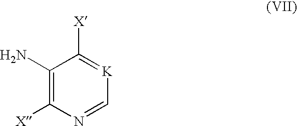
wherein X′ and X″ are independently halo.

11. A process according to claim 10 comprising conducting said contacting steps in a polar solvent.

12. A process according to claim 11 wherein said solvent is selected from the group consisting of water, n-butyl acetate, dimethylsulfoxide, 1-methyl-2-pyrrolidinone, methyl acetate, ethyl acetate and propyl acetate.

13. A process according to claim 12 further comprising including in one or more of said contacting steps a protic solvent.

14. A process according to claim 13 wherein the compound of formula (VI) is prepared by a process comprising selectively deprotecting a compound of formula (VIII): 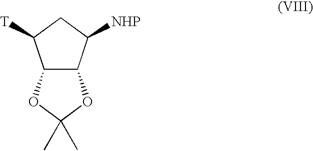
where P is a protecting group.

15. A process according to any of claims 1 to 6 wherein the compound of formula (I) is obtained in substantially pure form.

16. A process for the preparation of a compound of formula (V): 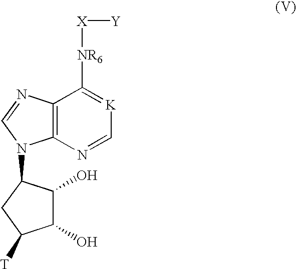
wherein:
K is N, N→O or CH;
R₆ is hydrogen, alkyl, allyl, 2-methylallyl, 2-butenyl or cycloalkyl;
X is 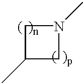
where the nitrogen of the ring of X is substituted by Y;
Y is hydrogen, alkyl, optionally substituted aralkyl, optionally substituted aryl, optionally substituted heterocyclyl or optionally substituted heterocyclylalkyl;
n and p are independently 0, 1, 2 or 3, provided that the sum of n and p is at least 1;
T is hydrogen, alkyl, acyl, thioacyl, halo, carboxyl, N(R₁)(R₂)C(═O), N(R₁)(R₂)C(═S) or R₃O—CH₂;
R₁, R₂ and R₃ are independently hydrogen, alkyl or cycloalkyl;
comprising:
contacting a compound of formula (I) 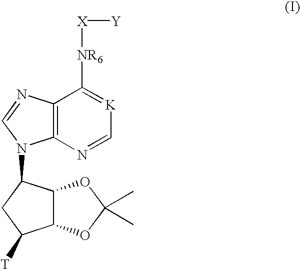
in the presence of an organic solvent, with water containing at least two equivalents of an acid to provide an aqueous medium containing the compound of formula (V) and an organic medium containing organic impurities;
adjusting th pH of the aqueous medium to a basic pH; and
isolating the compound of formula (V) from said aqueous medium.

17. A process according to claim 16 wherein said acid is selected from the group consisting of HCl, HBr, H₂SO₄, HNO₃ and acetic acid.

18. A process according to claim 17 wherein the compound of formula (V) is obtained in substantially pure form.

19. A process according to claim 18 wherein isolating comprises extracting the compound of formula (V) from said aqueous medium with an organic solvent.

20. A process according to claim 19 further comprising:
replacing said extraction solvent with a crystallization solvent; and
crystallizing the compound of formula (V) from said crystallization solvent.

21. A process according to claim 20 wherein said crystallization solvent is selected from the group consisting of acetonitrile, ethyl acetate, methanol, ethanol, isopropanol, butanol, or a combination thereof.

22. A process according to claim 21 wherein crystallizing the compound of formula (V) provides crystals having an average particle diameter of about 20 μm or less.

23. A process for the preparation of a compound of formula (VIII): 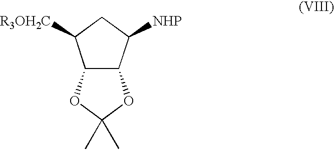
where P is a protecting group and R₃ is alky;
comprising protecting a compound of formula (ii): 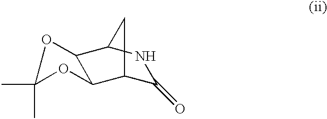
to provide a compound of formula (iii): 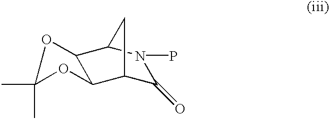
contacting the compound of (iii) with a reducing agent to provide a compound of formula (iv): 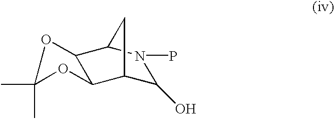
and alkylating the compound of formula (iv) to provide the compound of formula (VIII).

24. A process according to claim 23 wherein R₃ is methyl and the protecting group is tert-butyloxycarbonyl.

25. A process according to claim 24 wherein the reducing agent is selected from the group consisting of lithium borohydride and sodium borohydride; and
alkylating comprises contacting the compound of formula (iv) with an alkylating agent selected from the group consisting of CH₃OS(O)₂OCH₃, CH₃I, CH₃Br and CH₃Cl, in the presence of an acid scavenger.

26. A process for the preparation of a compound of formula (IV):
R₆NH—X—Y (IV)
wherein:
R₆ is hydrogen, alkyl or cycloalkyl;
X is 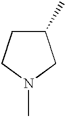
where the nitrogen of the ring of X is substituted by Y;
Y is optionally substituted aryl or optionally substituted heterocyclyl;
comprising contacting a compound of formula (vii): 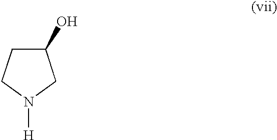
with a compound of formula Y—Z wherein Z is a halogen, in the presence of a first base to provide a compound of formula (ix): 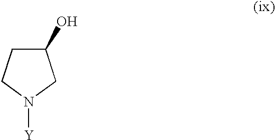
contacting the compound of formula (ix) with a sulfonating agent in the presence of a second base to provide a compound of formula (x): 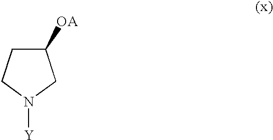
wherein —OA is a sulfonate ester;
contacting the compound of formula (x) with benzylamine to provide a compound of formula (xi): 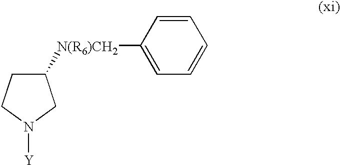
and hydrogenating the compound of formula (xi) in the presence of a hydrogenation catalyst to provide the compound of formula (IV).

27. A process according to claim 26 wherein:
R₆ is hydrogen;
Y is 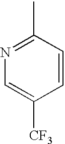
Z is Cl;
A is selected from the group consisting of methanesulfonyl, trifluorosulfonyl, p-toluenesulfonyl, and benzenesulfonyl;
the first base is selected from the group consisting of Li₂CO₃, K₂CO₃, Na₂CO₃, Cs₂CO₃, NaOH, KOH, and LiOH;
the second base is a tertiary amine; and
the hydrogenation catalyst is selected from the group consisting of palladium on carbon and palladium hydroxide on carbon.

28. A process for the preparation of a compound of formula (IV):
R₆NH—X—Y (IV)
wherein:
R₆ is hydrogen, alkyl or cycloalkyl;
X is 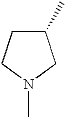
where the nitrogen of the ring of X is substituted by Y;
Y is optionally substituted aryl or optionally substituted heterocyclyl;
comprising contacting a compound of formula (xii): 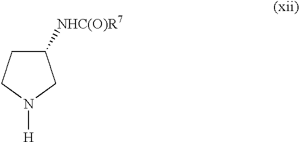
where R⁷ is an optionally substituted alkyl or aryl group,
with a compound of formula Y—Z wherein Z is a halogen, in the presence of a base to provide a compound of formula (xiii): 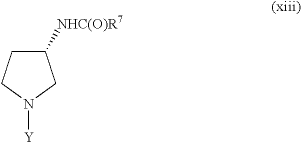
and contacting the compound of formula (xiii) with an acid to provide a compound of formula (IV).

29. A process according to claim 28 wherein:
R₆ is hydrogen;
Y is 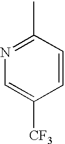
Z is Cl;
the base is a tertiary amine; and
the acid is selected from the group consisting of hydrochloric acid, hydrobromic acid, sulfuric acid, phosphoric acid, nitric acid, acetic acid, formic acid, trifluoroacetic acid, propionic acid, and methanesulfonic acid.

30. A process for the preparation of a compound of formula (IV):
R₆NH—X—Y (IV)
wherein:
R₆ is hydrogen, alkyl or cycloalkyl;
X is 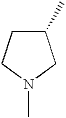
where the nitrogen of the ring of X is substituted by Y;
Y is optionally substituted aryl or optionally substituted heterocyclyl;
comprising contacting a compound of formula (xiv): 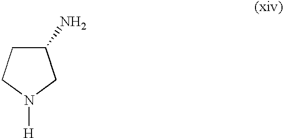
with a compound of formula Y—Z wherein Z is a halogen, in the presence of a base to provide the compound of formula (IV).

31. A process according to claim 30 wherein:
R₆ is hydrogen;
Y is 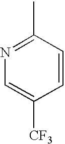
Z is Cl; and
the base is selected from the group consisting of Li₂CO₃, K₂CO₃, Na₂CO₃, Cs₂CO₃, NaOH, KOH, and LiOH.

32. An acid addition salt of the compound of formula (I): 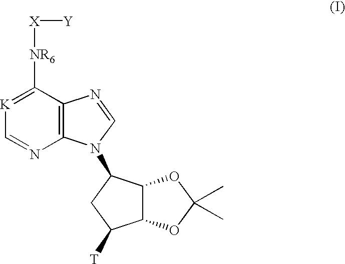
wherein:
K is N, N→O or CH;
R₆ is hydrogen, alkyl, allyl, 2-methylallyl, 2-butenyl or cycloalkyl;
X is 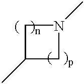
where the nitrogen of the ring of X is substituted by Y;
Y is hydrogen, alkyl, optionally substituted aralkyl, optionally substituted aryl, optionally substituted heterocyclyl or optionally substituted heterocyclylalkyl;
n and p are independently 0, 1, 2 or 3, provided that the sum of n and p is at least 1;
T is hydrogen, alkyl, acyl, thioacyl, halo, carboxyl, N(R₁)(R₂)C(═O), N(R₁)(R₂)C(═S) or R₃O—CH₂; and
R₁, R₂ and R₃ are independently hydrogen, alkyl or cycloalkyl.

33. An acid addition salt of the compound according to claim 32 wherein the acid is selected from the group consisting of hydrochloric acid, methanesulfonic acid, oxalic acid, D-tartaric acid, succinic acid, L-tartaric acid, fumaric acid, formic acid, benzoic acid, dibenzoyl-D-tartrate, dibenzoyl-L-tartrate, di-p-toluoyl-D-tartrate, di-p-toluoyl-L-tartrate, (−)-mandelic acid, maleic acid, acetic acid, glycolic acid, salicylic acid; D-gluconic acid, p-toluenesulfonic acid, phosphoric acid, and hydrobromic acid.

34. An acid addition salt of the compound according to claim 32 or 33 wherein the acid is methane sulfonic acid.

35. An acid addition salt of the compound according to claim 34 wherein the compound has formula (I-i):

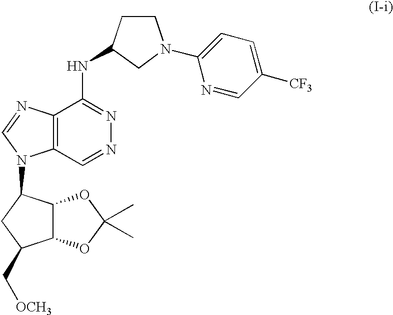

36. An acid addition salt of the compound of formula (VI): 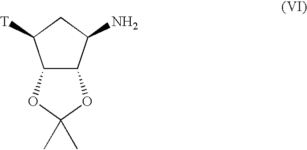
wherein T is hydrogen, alkyl, acyl, thioacyl, halo, carboxyl, N(R₁)(R₂)C(═O), N(R₁)(R₂)C(═S) or R₃O—CH₂.

37. An acid addition salt of the compound according to claim 36 wherein the acid is selected from the group consisting of hydrochloric acid, oxalic acid, D-tartaric acid, succinic acid, L-tartaric acid, fumaric acid, formic acid, benzoic acid, dibenzoyl-D-tartrate, dibenzoyl-L-tartrate, di-p-toluoyl-D-tartrate, di-p-toluoyl-L-tartrate, (−)-mandelic acid, maleic acid, acetic acid, glycolic acid, salicylic acid, D-gluconic acid, methanesulfonic acid, p-toluenesulfonic acid, phosphoric acid, and hydrobromic acid.

38. An acid addition salt of the compound according to claim 37 wherein T is R₃O—CH₂ and the acid is oxalic acid.

39. A process according to claim 1 which is substantially concatenated.

40. A process according to claim 7 wherein the compound of formula (I) is obtained in substantially pure form.

41. A process according to claim 10 wherein the compound of formula (I) is obtained in substantially pure form.

42. A process according to claim 14 wherein the compound of formula (I) is obtained in substantially pure form.
